(19) United States
(12) Patent Application Publication
(10) Pub. No.: US 2023/0218637 A1
(43) Pub. Date: Jul. 13, 2023

(54) COMPOSITIONS AND METHODS FOR DIAGNOSING, PREVENTING, AND TREATING AMYOTROPHIC LATERAL SCLEROSIS IN PATIENTS WITH HYPOFUNCTIONAL ANTI-TRYPSIN ACTIVITY

(71) Applicant: Motor Life Sciences, LLC, Stow, OH (US)

(72) Inventors: Jaclyn Dunphy, Cambridge, MA (US); Thomas Charles Stenger, Stow, OH (US); Mary Louise Stenger, Stow, OH (US)

(21) Appl. No.: 17/929,609

(22) Filed: Sep. 2, 2022

Related U.S. Application Data
(63) Continuation of application No. PCT/US2021/020537, filed on Mar. 2, 2021.
(60) Provisional application No. 62/984,157, filed on Mar. 2, 2020.

(51) Int. Cl. A61K 31/575; C12Q 1/37; C12Q 1/6883; A61P 25/00

(52) U.S. Cl. CPC A61K 31/575; C12Q 1/37; C12Q 1/6883; A61P 25/00; C12Q 2600/156; C12Q 2600/106; G01N 2333/976; G01N 2333/966; G01N 2800/52; G01N 2333/8125

(57) ABSTRACT

The disclosure provides biomarkers for ALS. The disclosure also provides various methods of using the biomarkers, including methods for diagnosis of ALS, methods of determining predisposition to ALS, methods of monitoring progression/regression of ALS, methods of assessing efficacy of treatment modalities for treating ALS, methods of screening compositions for activity in modulating biomarkers of ALS, methods of treating ALS, as well as other methods based on biomarkers of ALS. The disclosure also provides various methods of treating other diseases, disorders, and conditions. One method includes analyzing levels of alpha-1 antitrypsin (A1AT, also known as AAT or PI), encoded by the serpinA1 gene or protein as a biomarker indicative of ALS. The levels or concentrations of the biomarkers can be used to determine the onset of ALS, monitor the progression of ALS, or monitor the progression of a treatment for ALS.

### CROSS-REFERENCE TO RELATED APPLICATIONS

This application is a continuation of International Application No. PCT/US2021/020537 filed Mar. 2, 2021, which claims the benefit of U.S. Provisional Application No. 62/984,157 filed Mar. 2, 2020, which is hereby incorporated by reference in its entirety for all purposes.

### FIELD OF THE INVENTION

The present invention relates generally to the field of human neurodegenerative diseases, including amyotrophic lateral sclerosis (“ALS”) and to methods of diagnosing or predicting ALS and identifying potential ALS therapeutic agents. The invention has applications in the fields of neurological medicine.

### BACKGROUND

Amyotrophic lateral sclerosis (ALS), also called Lou Gehrig's disease, is a progressive, fatal neurological disease affecting nerve cells in the brain and spinal cord. ALS belongs to a class of disorders known as motor neuron diseases. ALS occurs when motor neurons in the brain and spinal cord degenerate. Clinical characterization includes progressive muscle weakness, muscle atrophy and eventually paralysis. Currently there is no cure for ALS, and patients have an average life expectancy of two to five years after diagnosis. Current therapies slow down disease progression, with one expanding life expectancy by a mere three months.

It is estimated that at least 16,000 Americans are living with ALS at any point in time. Roughly 5,000 people are diagnosed with ALS per year, with about 15 new cases per day. 90 percent of ALS cases are sporadic, with unknown cause(s) of origin. The other ten percent of cases are familial, where the disease is inherited from a family member. Currently there are several familial gene mutations (SOD1, TARDBP, FUS, UBQLN2, C9ORF72) with other factors that are associated with the typical clinical phenotype. It is useful to develop new modalities to diagnose, treat, and prevent ALS and the symptom(s) thereof.

SerpinA1 belongs to the serpin superfamily and produces the alpha-1 anti-trypsin (A1AT) protein which inhibits target enzymes including trypsin. Serpins are conformationally labile and disease-linked mutations result in protein misfolding and protein aggregation. Expression of SerpinA1 protein and corresponding activity has been shown to be elevated in both the frontal cortex and cerebellum of both sporadic and inherited forms of ALS. (Ebbert et al, 2017). Outside the brain, SerpinA1 was found to be elevated by 80% in a muscle biopsy of an astronaut who was experiencing muscle wasting and weakness upon return from a 6-month spaceflight to the International Space Station (Capri et al, 2019). However, the role of SerpinA1 in the complex biology of ALS is still unclear.

The SerpinA1 expression product A1AT blocks neutrophil elastase. Neutrophil elastase hyperactivity is known to cause tissue damage. For example, hyperactivity of neutrophil elastase in the lung leads to dysfunction, which can cause emphysema. A1AT deficiency is a leading cause of early-onset emphysema. However, the role of neutrophil elastase in neurological diseases, such as the complex biology of ALS, is not yet understood.

SerpinA1 mutations have been known to cause chronic obstructive pulmonary disease (COPD) and liver disease, due to a loss-of-function and damage-causing protein-misfolding gain-of-function, respectively. In the former scenario, chronic inflammation results due to loss-of-function SerpinA1 mutants that no longer properly inhibit trypsin leading to uncontrolled levels of trypsin activity in tissues. In the latter scenario, gain-of-function SerpinA1 mutants reduce the activity of enzymes in the unfolded protein response (UPR) pathway, such as trypsin, leading to misfolded protein aggregates in the endoplasmic reticulum, disrupting physiological autophagy processes that would normally halt further protein aggregation (Marciniak et al, 2016). However, the role of these SerpinA1 mutations in neurological diseases, such as the complex biology of ALS, is not yet fully understood. Modified serpinA1 in cerebrospinal fluid (CSF) has also been suggested as an early biomarker for differentiation between Parkinson patients with dementia (PDD) or without dementia (PD). Wormser et al. demonstrated that A1AT levels were significantly decreased in the CSF of patients with ALS. (Wormser, Uri et al. “Reduced levels of alpha-1-antitrypsin in cerebrospinal fluid of amyotrophic lateral sclerosis patients: a novel approach for a potential treatment.” Journal of neuroinflammation vol. 13, 1 131. 1 Jun. 2016, doi:10.1186/s12974-016-0589-4. However, these mutations are not currently considered primary drivers of disease nor are they currently among the common genetic mutations or single-nucleotide polymorphisms used for diagnosing either familial or sporadic ALS. For example, genotyping for mutations in A1AT are currently confined to patients presenting with A1AT deficiency-like symptoms generally affecting the lung (such as COPD) or liver (such as jaundice) or those with a genetic pre-disposition for the disease (offspring of patients with A1AT deficiency).) In addition, the functional activity of the patient samples was not assessed in the study and decreased levels of the protein have been proposed as indicators of pathology rather than drivers of disease. In another study, showed an increase in differentially methylated cytosine in the frontal cortex and cerebellum in patients with C9orf72 or sporadic ALS (Ebbert, Mark T W et al. “Conserved DNA methylation combined with differential frontal cortex and cerebellar expression distinguishes C9orf72-associated and sporadic ALS, and implicates SERPINA1 in disease.” Acta neuropathologica vol. 134,5 (2017): 715-728. doi:10.1007/s00401-017-1760-4). This led to the over-expression of serpinA1 protein, particularly in the brain. Although elevated neutrophil elastase activity and/or altered serpinA1 expression and activity have been correlated with neurodegenerative diseases, these changes are generally considered to be correlative rather than causative. Accordingly, the role of serpinA1 mutations in driving neurological diseases are their value in diagnostic panels remains unclear. Notably, serpinA1 biological pathways have not been explored as a target for therapeutic intervention in ALS.

Therapies directed to SerpinA1 biology including modulation of neutrophil elastase may be useful but are currently lacking in the context of the treatment of complex neurological diseases, such as ALS. Accordingly, still needed in the field are treatments and associated diagnostic methods directed to SerpinA1 biology and neutrophil elastase and their role in neurological diseases. Also needed are improved diagnostic approaches directed to SerpinA1 biology in neurological diseases, including the role of serpinA1 mutations in driving neurological diseases.

### SUMMARY

Provided for herein is a method of treating amyotrophic lateral sclerosis (ALS) in a human subject in need thereof comprising administering to the subject an agent in an amount effective to i) increase an alpha-1 anti-trypsin (A1AT) activity and/or ii) decrease a serine protease activity in a target cell, tissue or organ of the subject, wherein the subject is characterized as having insufficient alpha-1-anti-trypsin activity in the target cell, tissue or organ.

Also provided for herein is a pharmaceutical composition comprising a) an agent in an amount effective to i) increase an alpha-1 anti-trypsin (A1AT) activity and/or ii) decrease a serine protease activity in a target cell, tissue or organ of a subject, wherein the subject is characterized as having insufficient alpha-1-anti-trypsin activity in the target cell, tissue or organ and b) a pharmaceutically acceptable carrier. In some aspects, the human or animal subject is characterized as having ALS or at risk of developing ALS.

Also provided for herein is a method of treating ALS or a symptom thereof in a human subject, the method comprising: a. obtaining or having obtained a biological sample selected from cerebrospinal fluid, blood, urine, gastrointestinal or fecal matter, saliva, tears or tissue from the subject; b. assessing or having assessed the biological sample for serpinA1/A1AT levels and/or alpha-1 antitrypsin activity, wherein the activity comprises serine protease inhibitor activity; c. characterizing from the assessment whether the subject has a deficiency in an alpha-1 antitrypsin activity, optionally wherein the subject is characterized as having the deficiency in an alpha-1 antitrypsin activity when (1) a A1AT protein level below a threshold range of 0.9 and 2.3 g/L is detected in the biological sample, (2) an A1AT activity below a threshold activity correlating to activity of a reference sample comprising wild-type A1AT protein level in a range of 0.9 and 2.3 g/L is detected in the biological sample, and/or (3) an neutrophil elastase protein level above a threshold level correlating to neutrophil elastase protein levels in a reference sample comprising wild-type A1AT protein level in a range of 0.9 and 2.3 g/L is detected in the biological sample; and d. administering to the subject an agent that inhibits serine protease activity in an amount effective to treat ALS when the subject is characterized as having the deficiency in an alpha-1 antitrypsin activity.

Also provided for herein is a method of treating ALS or a symptom thereof in a human subject, the method comprising: a. obtaining or having obtained a biological sample selected from cerebrospinal fluid, blood, urine, gastrointestinal or fecal matter, saliva, tears or tissue from the subject; b. assessing or having assessed the biological sample for serine protease activity, optionally wherein the subject is characterized as having a deficiency in an alpha-1 antitrypsin (serpinA1) activity, wherein the activity comprises serine protease inhibitor activity; c. characterizing from the assessment whether the subject has an excess of serine protease activity, optionally wherein the subject is characterized as having the excess serine protease activity when serine protease activity above a threshold activity correlating to serine protease activity in a reference sample comprising wild-type A1AT protein level in a range of 0.9 and 2.3 g/L is detected in the biological sample; and d. administering to the subject an effective dose of an agent that increases A1AT expression or activity in an amount effective to treat ALS when the subject is characterized as having the excess of serine protease activity.

In some aspects, the characterization of the activity is with reference to a reference level in an organ or tissue. In some aspects, the organ or tissue is blood, cerebrospinal fluid, muscle, or gastrointestinal tract.

In some aspects, the symptom is stumbling; slurred speech; difficulty swallowing; muscle cramps; worsening posture; difficulty with holding the head up; muscle stiffness; difficulty walking; tripping and/or falling; hand weakness; twitching in one's arms, shoulders or tongue; inappropriate crying, laughing or yawning; or cognitive and/or behavior problems.

In some aspects, the A1AT expression or activity is increased in the brain, liver immune system and/or gastrointestinal tract.

In some aspects, the agent comprises a cell therapy. In some aspects, the cell therapy comprises an engineered cell engineered to express a functional A1AT protein. In some aspects, the cell therapy comprises a stem cell, neuron, glial cell, muscle cell, pancreas cell, hepatocyte, immune cell, bacterial cell, or archaeal cell.

In some aspects, the agent comprises an antibody, an enzyme activator, small molecule, or mRNA formulated into lipid nanoparticles.

In some aspects, the agent comprises a replacement therapy. In some aspects, the replacement therapy comprises an A1AT replacement therapy. In some aspects, the replacement therapy comprises administration of a functional enzyme. In some aspects, the replacement therapy comprises a purified A1AT protein, optionally wherein the purified A1AT protein comprises Aralast NP. In some aspects, the replacement therapy comprises editing a genome of the subject to produce a functional enzyme, optionally wherein the genome editing is Crispr/Cas9-mediated. In some aspects, the replacement therapy comprises an engineered cell engineered to express a functional enzyme.

Also provided for herein is a method comprising administering to a human subject an agent comprising a pharmaceutically acceptable vehicle and exogenous antibody or binding fragment thereof that binds selectively to all or part of mutant A1AT, wherein administering the antibody or binding fragment thereof treats or prevents ALS or a symptom thereof.

Also provided for herein is a method comprising: administering to a human subject an agent comprising a pharmaceutically acceptable vehicle and exogenous antibody or binding fragment thereof that binds selectively to all or part of endogenous A1AT, wherein administering the antibody or binding fragment thereof treats ALS or a symptom thereof. In some aspects, the antibody is a monoclonal, polyclonal, chimeric or humanized antibody. In some aspects, the antibody binds at least 5 contiguous amino acids of a serpinA1 polypeptide. In some aspects, the antibody binds at least 5 contiguous amino acids of a serpinA1 variant polypeptide expressed by the subject.

Also provided for herein is a pharmaceutical composition comprising an enzyme activator that increases A1AT expression or activity when administered to a human subject in an amount effective to treat or prevent ALS or a symptom thereof.

Also provided for herein is a pharmaceutical composition comprising a small molecule having a molecular weight less than about 5 kiloDaltons in an amount effective to increase A1AT expression or activity when administered to a human subject.

Also provided for herein is a pharmaceutical composition comprising mRNA formulated into a lipid nanoparticle in an amount effective to increase A1AT expression or activity when administered to a human subject.

Also provided for herein is a pharmaceutical composition comprising an antibody in an amount effective to increase A1AT expression or activity when administered to a human subject.

Also provided for herein is a pharmaceutical composition of a pharmaceutically acceptable vehicle and an effective amount of an antibody or binding fragment thereof that binds selectively to a A1AT wild-type polypeptide or A1AT variant polypeptide. In some aspects, the antibody binds at least 5 contiguous amino acids of serpinA1 polypeptide. In some aspects, the antibody binds at least 5 contiguous amino acids of a serpinA1 variant polypeptide expressed by the subject. In some aspects, the antibody reduces serpinA1 activity in a human subject.

Also provided for herein is a pharmaceutical composition of a pharmaceutically acceptable vehicle and an effective amount of an inhibitory nucleic acid thereof that reduces a level of a serpinA1 variant polypeptide.

Also provided for herein is a pharmaceutical composition of a pharmaceutically acceptable vehicle and an effective amount of engineered bacteria to reduce a level of a serpinA1 variant polypeptide in a human subject.

Also provided for herein is a pharmaceutical composition of a pharmaceutically acceptable vehicle and an effective amount of engineered bacteria to reduce serine protease activity in the gastrointestinal tract of a human subject.

In some aspects, the agent comprises an autophagy activator, a mitophagy activator, a neutrophil elastase inhibitor, or a combination thereof, optionally wherein: a. the autophagy activator is independently selected from the group consisting of: carbamazepine (Tegretol), Akebia saponin D, metformin, rapamycin, resveratrol, retinoic acid, and valproic acid; b. the mitophagy activators is independently selected from the group consisting of celastrol (Tripterin), Urolithin A; and c. the neutrophil elastase inhibitor is independently selected from the group of ursolic acid, Sivelestat, Alvelestat, sirtinol, and triterpene derived compounds.

In some aspects, the agent comprises an effective amount of Alvelestat. In some aspects, the agent comprises an oral dose of Alvelestat, wherein the dose administered is at least about 120 mg, suitable to be administered one or more times per day.

In some aspects, the agent comprises an alpha-tocopherol (Vitamin E) analog.

In some aspects, the agent comprises a triterpene. In some aspects, the triterpene administered comprises ursodiol or derivative. In some aspects, the triterpene administered comprises ursolic acid or derivative. In some aspects, the triterpene administered comprises a pentacyclic triterpenoid or derivative. In some aspects, the methods or pharmaceutical compositions further comprise a second agent that is capable of restoring gut barrier function.

Also provided for herein is a method of identifying a compound capable of modulating a serine protease inhibitor activity, comprising the steps of (a) identifying one or more functional groups capable of interacting with a serpinA1; and (b) identifying a scaffold which is capable of orienting the one or more functional groups identified in (a) in a suitable orientation for interacting with the serpinA1.

Also provided for herein is a pharmaceutical composition comprising any one of the compounds identified herein in an amount effective to increase serine protease inhibitor activity when administered to a human subject. In some aspects, the serpinA1 comprises a variant serpinA1 polypeptide.

Also provided for herein is a method for identifying a subject suitable for treatment of ALS comprising: a. measuring a first concentration of an alpha-1 anti-trypsin in a biological sample from the subject; b. measuring a second concentration of a neurofilament in the sample; and c. determining a value comprising the ratio of the first concentration to the second concentration, wherein the value above a threshold value selects the subject for treatment. In some aspects, the biological sample comprises cerebrospinal fluid, blood, urine gastrointestinal or fecal matter, saliva, tear, or tissue. In some aspects, the subject has an impaired liver activity or function. In some aspects, the method of determining impaired liver activity comprises the step of measuring a steroid hormone level, bile acid production, or production of a cholesterol, lipid, HDL, LDL, triglyceride or cytokine.

Also provided for herein is a biomarker assay for diagnosis of ALS in a human subject, comprising means for determining an insufficient level of serpinA1 protein in the subject.

Also provided for herein is a biomarker assay for diagnosis of ALS in a human subject, comprising means for determining a variant serpinA1 protein in the subject.

Also provided for herein is a biomarker assay for diagnosis of ALS in a human subject, comprising means for determining serpinA1 protein aggregates in the subject.

Also provided for herein is a biomarker assay for diagnosis of ALS in a human subject, comprising means for determining serpinA1-neurofiliment protein aggregates in the subject.

Also provided for herein is a biomarker assay for diagnosis of ALS in a human subject, comprising means for determining trypsin cleavage products in the subject.

Also provided for herein is a method for identifying a human subject for treatment of ALS comprising using the biomarker assay of any one of biomarker assays described herein with a biological sample from a subject, and determining a predictive value, which if greater than a certain threshold value identifies the subject as suitable for treatment. In some aspects, the biological sample comprises blood, plasma, derived from peripheral blood mononuclear cell or collected from brain tissue, muscle, liver tissue, or gastrointestinal tract tissue.

Also provided for herein is a method of treating ALS in a human subject, comprising administering an agent that increases alpha-1 anti-trypsin (A1AT) activity to the subject wherein the subject is characterized as having hypofunctional alpha-1-anti-trypsin activity.

Also provided for herein is a method of treating ALS in a human subject, comprising administering an agent that decreases serine protease activity to the subject wherein the subject is characterized as having hypofunctional alpha-1-anti-trypsin activity. In some aspects, the subject has a level of serpinA1 function less than a reference level in an organ or tissue. In some aspects, the subject has a level of serine protease activity greater than a reference level in an organ or tissue. In some aspects, the organ or tissue is blood, cerebrospinal fluid, muscle, or gastrointestinal tract. In some aspects, the method comprises the step of administering to the subject an effective amount of an agent that modulates an antitrypsin pathway. In some aspects, the agent comprises Prolastin-C, Aralast NP, Zemaira, Glassia or Trypsone. In some aspects, the agent is orally delivered, inhaled, infused intravenously, applied transdermal or injected intradermally, intramuscularly, intravenously, or intracranially.

### DETAILED DESCRIPTION

Provide herein are methods of treating amyotrophic lateral sclerosis (ALS) that include modulation of SerpinA1 biological pathways, such as modulation of neutrophil elastase activity. A1AT, the expression product of serpinA1, blocks neutrophil elastase and is useful for preventing pathological tissue disruption. Accordingly, methods described herein can include administering to in a human subject in need thereof an agent in an amount effective to i) increase an alpha-1 anti-trypsin (A1AT) activity and/or ii) decrease a serine protease activity in a target cell, tissue or organ of the subject, wherein the subject is characterized as having insufficient alpha-1-anti-trypsin activity in the target cell, tissue or organ. Also provided herein are pharmaceutical compositions (e.g., therapeutic agents) directed to the same that can include a) an agent in an amount effective to i) increase an alpha-1 anti-trypsin (A1AT) activity and/or ii) decrease a serine protease activity in a target cell, tissue or organ of a subject, wherein the subject is characterized as having insufficient alpha-1-anti-trypsin activity in the target cell, tissue or organ and b) a pharmaceutically acceptable carrier.

Neurodegenerative diseases (e.g., ALS) can be treated by modulating dysregulated neutrophil elastase activity, provided for herein are therapies that modulate (e.g., block, inhibit, or regulate) neutrophil elastase activity to ameliorate neurodegenerative diseases. Alvelestat is a neutrophil elastase inhibitor that is being tested in clinical trials for alpha-1 antitrypsin deficiency and can be used as an agent to modulate neutrophil elastase activity. In addition, neurodegenerative diseases (e.g., ALS) can be treated through treating A1AT deficiency, such as through an A1AT replacement therapy. A1AT replacement therapy can include, but is not limited to, administration of a functional enzyme, such as the human alpha1-protease inhibitor enzyme therapy Aralast NP (Baxter), genetic replacement therapy such as Crispr/Cas9 mediated introduction of functional serpinA1 (e.g., using Intellia's lipid nanoparticle virus therapy), ex vivo engineered cell therapy to ameliorate neurodegenerative diseases, or combinations thereof. The Crispr/Cas9 gene-editing lipid nanoparticle therapy developed by Intellia has been demonstrated to restore physiological levels of alpha-1 anti-trypsin protein in the liver of non-human primates after a single dose. The “PiZ-variant” (see Table 1 below) is the most common genetic mutation underlying alpha-1 anti-trypsin deficiency, but other variants can result in alpha-1 anti-trypsin hypofunction. Therefore, a gene therapy that replaces hypofunctional serpinA1 protein (including but not limited to the “PiZ-variant”) can be used to treat patients suffering from neuroinflammatory diseases such as ALS. Ex vivo cell therapy that includes increasing expression and/or activity of serpinA1 in cells followed by introducing the engineered cells into the body can be used to treat patients suffering from neuroinflammatory diseases such as ALS. Cells engineered to express functional serpinA1 (e.g., via Crispr/Cas9-mediated genome engineering, electroporation, transfection, or other methods of introducing DNA or proteins into cells that are known in the art), can be derived from a patient or orthogonal donor. Cells suitable for ex vivo engineering include, but are not limited to, stem cells, hepatocytes, myocytes, neurons, astrocytes, oligodendrocytes, microglia, lymphocytes, red blood cells, cells of the pancreas, and monocytes. Alternatively, cell therapy can include bacterial or archaeal cells that make up the human microbiome. For example, bacteria can be engineered to reduce protease activity in the gastrointestinal tract through (1) the expression of an anti-trypsin enzyme derived from humans (such as serpinA1) or other organism, and/or (2) via the production and secretion of a protease inhibitor compound such as the triterpene ursolic acid. Administration of the bacterial therapy can reduce inflammation affecting muscles, neurons and nerves distributed throughout the body, including in the brain, gastrointestinal tract, and neuromuscular junction. In addition, A1AT replacement therapies can be used in combination with treatments (e.g., agents) that modulate neutrophil elastase activity.

Therapies that can be useful for treating neurodegenerative diseases (e.g., ALS) also include, but are not limited to, therapies known for the treatment of diseases and conditions associated with SerpinA1 mutations. Currently available treatments of these indications (lung or liver dysfunction and genotyped to demonstrate mutant A1AT alleles) include infusion of wild-type A1AT protein (replacement therapy, also known as augmentation therapy), including, but not limited to, Aralast NP, Zemaira, Glassia, and Prolastin-C. Therapies that can be useful for treating neurodegenerative diseases (e.g., ALS), such as treating tissue degeneration experienced by ALS patients, also include, but are not limited to, therapies known for the treatment of diseases and conditions associated with insufficient anti-trypsin activity. For example, provided herein is a method of administering therapeutic agents (e.g., drugs) designed for patients with genetic alpha-1 antitrypsin deficiencies, such as for ALS patients who have elevated serine protease activity.

The present invention relates to methods and uses for identifying candidate molecules, compounds or therapies for treating neurological diseases such as ALS. The invention provides a method for treating a medical condition, disease, or disorder mediated by serine protease inhibitors, such as a misfolded form of serine protease inhibitor (serpinA1), in a subject in need of treatment. In certain embodiments, the invention provides methods of treating amyotrophic lateral sclerosis by targeting disease-specific epitopes, and compositions that target such epitopes. As an illustrative non-liming example, mutations in serpinA1 may lead to protein misfolding that can reveal disease specific epitopes. that Antibodies can be generated and/or constructed that target such disease specific epitopes. Accordingly, the invention also provides antibodies that bind to misfolded serpinA1, and not on the molecular surface of native serpinA1. For example, hybridoma cell line, ATZ11, produces an IgG antibody that binds to a mutant form of A1AT protein (the PiZ mutation) (Wallmark et al, 1984, herein incorporated by reference for all purposes) but not the wild-type serpinA1 protein. This antibody has been used to detect carriers of PiZ alleles via immunochemical means instead of genetic testing.

Early detection, such as for dementia in Parkinson's disease, is generally performed for preventive therapeutic approaches. SerpinA1/A1AT can be genotyped and assessed for mutation status. SerpinA1/A1AT mutations and symptoms can indicate the need for treatment. Such mutation monitoring can be performed together with or separate from assessing the level of protease inhibitor activity compared to the level of protease activity. Even in the absence of genetic mutation, assessing the level of protease inhibitor activity compared to the level of protease activity can indicate an imbalance between the two processes and provide an additional indication for the need for a treatment. Pathological changes (e.g., altered expression or mutant sequence) in serpinA1 or proteases (e.g., trypsin, neutrophil elastase) can be those that are primary drivers of cell death associated with these diseases. For example, specific serpinA1 mutations that have been determined to drive or predominantly contribute to ALS symptoms can be used to assess a need for treatment. Administration of a therapy, such as A1AT replacement therapies and/or other agents that modify SerpinA1 biology described herein, can be administered following such an assessment.

Methods of assessing SerpinA1/A1AT mutation status and/or protease activity are generally known to those of skill in the art and may be used. Methods of determining serpinA1 abundance and activity are also generally known to those of skill in the art and may be used. A newly developed nanoscale method for the detection of serpinA1 based on automated capillary isoelectric focusing (LIEF) was conducted by Halbgebauer et. al. (Halbgebauer, Steffen et al. “Modified serpinA1 as risk marker for Parkinson's disease dementia: Analysis of baseline data.” Scientific reports vol. 6 26145. 17 May. 2016, doi:10.1038/srep26145). A clinical sample was collected for one-hundred and two (102) subjects including neurologically healthy controls (CON), PD and PDD patients was investigated. Seven serpinA1 isoforms of different charge were detected in CSF from all three diagnostic groups. The mean CSF signals of the most acidic serpinA1 isoform differed significantly (p<0.01) between PDD (n=29) and PD (n=37) or CON (n=36). Patients above the cut-off of 6.4 have a more than six times higher risk for an association with dementia compared to patients below the cut off. Notably, the above assay is directed to serpinA1 isoforms whose functional or mutation status is not known. Accordingly, as provided herein, serpinA1 CIEF-immunoassays can be used to assess and/or predict cognitive impairment in PD and ALS patients through monitoring specific serpinA1 mutations (e.g., mutations that have been determined to drive or predominantly contribute to ALS symptoms) and/or determining serpinA1 abundance and activity as it correlates to neurological diseases. As an illustrative non-limiting example, a patient with mutant serpinA1 may demonstrate high levels of dysfunctional serpinA1 protein and the methods provided herein can quantify the amount and activity of serpinA1 protein for the purposes of determining whether a patient would benefit from a serpinA1 modulating therapy.

In addition to diagnostic panels assessing SerpinA1/A1AT mutation status, panels can also include any of the approximately 40 genes on current ALS panels, including, but not limited to SOD1, TREM2, PSEN1, and MAPT, or combinations thereof.

As used herein, the term “sample” refers to an isolated biological material from the subject. The sample, includes but not limited to any suitable biomaterial, preferably comprises cells or metabolites obtained from a particular tissue or biological fluid. The sample can be isolated from any suitable tissue or biological fluid. In this regard, the sample includes but is not limited to blood, serum, plasma, urine, CSF, ocular fluid, tears, saliva, lumbar puncture (by ventricular puncture, cisternal puncture, or obtained by any suitable methods known in the art). As ALS affects the central nervous system, the sample is preferably from the central nervous system (CNS) (i.e., brain, spinal cord and/or cerebral spinal fluid) and are isolated from a tissue or biofluid thereof. In a particular embodiment of the present invention, the sample is isolated from the cerebrospinal fluid (CSF). CSF regulates brain extracellular fluid, allows the distribution of neuroactive substances, and removal of waste products generated by the brain. Many metabolites from neurons and glial cells from the CNS are secreted into CSF. In this way, CSF, generally more than other biological fluids, can directly reflect the metabolic state and biochemical contents of the brain.

The invention provides a method for treating a medical condition, disease, or disorder mediated by a misfolded form of serine protease inhibitor (serpinA1) in a subject in need of treatment. In one embodiment, the method comprises administering to the subject a composition comprising a pharmaceutically acceptable vehicle and an agent selected from (1) an exogenous antibody or fragment thereof that binds selectively to the misfolded form of serpinA1, and/or (2) an immunogen that elicits production of an endogenous antibody that binds selectively to the misfolded form of serpinA1, and/or (3) a nucleic acid sequence encoding (1) or (2). In certain embodiments, the invention provides methods of treating diseases including but not limited to: Alzheimer's Disease, Parkinson's Disease or amyotrophic lateral sclerosis by targeting amyotrophic lateral sclerosis disease-specific epitopes, and compositions that target such epitopes. The invention also provides antibodies that bind to monomeric or misfolded serpinA1, or aggregates and polymers of serpinA1, but not on the molecular surface of native serpinA1, such as those produced by ATZ11 hybridoma cells (Wallmark et al, 1984) or discovered by Miranda et al, 2010. In addition, the invention includes methods of diagnosing Alzheimer's Disease, Parkinson's Disease or amyotrophic lateral sclerosis in a subject. Also, the invention provides methods of identifying substances for the treatment or prevention of Alzheimer's Disease, Parkinson's Disease or amyotrophic lateral sclerosis and kits using the binding proteins of the invention.

Common variants for serpinA1 include rs17580 (PiS, Glu264Val), rs28929474 (PiZ, Glu342Lys), and rs6647, (PiM, a1a213). More than 70 other variants in this gene exist and can be assessed for their association with disease. Abundance of A1AT, including variants and/or wild-type alleles, can be assessed. Activity of A1AT, including variants and/or wild-type alleles, can be assessed. Abundance and activity of A1AT including variants and/or wild-type alleles, can be assessed. As an illustrative non-limiting example, and A1AT sequence can be determine to be wild-type or mutated and activity assessed, for example given “normal” levels of a mutant A1AT protein could result in a functional deficiency. Each of the serpinA1 sequence variants interacts with an electrophoretic field differently. The PiF protein, for example, moves “fast”, PiM proteins moves “medium”, PiS proteins move “slow”, and the PiZ allele moves “very slow”. The speed at which the serpinA1 protein moves through a gel can be used as a biomarker indicating a pathological variant of the gene.

Neurofilamentous conglomerates composed of neurofilament proteins and their cognate serpins are found in motor neurons (Chou, Samuel et al. “Serpin=serine protease-like complexes within neurofilament conglomerates of motoneurons in amyotrophic lateral sclerosis.” J Neurol Sci. 160 Suppl 1:S73-9 Oct. 1998, doi: 10.1016/s0022-510x(98)00202-0) The presence of inclusion bodies containing at least one neurofilament protein and a serpin protein in a patient-derived cell can be assessed, such as a biomarker for ALS. Such assessment can be in addition to or separate from assessing abundance and/or activity of A1AT. For example, assessment of inclusion bodies can be used where neither mutations in the serpin protein, nor abnormalities in a serpin-neurofilament interaction due to mutations in the serpin protein, have been determined. The invention provides methods of determining a diagnosis or prognosis of motor neuron disease in a mammal comprising determining the expression level of one or more proteins or polypeptides of the serpin system in a sample taken from a subject. Similarly, aberrant post-translational modification of the proteins or polypeptides as compared to a negative control indicates a diagnosis of disease.

Whole genome sequencing can be performed by commercial vendors from saliva samples provided by consumers or patients. Screening of the genome for genetic mutations known to cause or correlated with a variety of diseases can be provided as a service. It is possible to detect mutations in the serpinA1 sequence and report the results to consumers/patients. The full genome raw sequence data (as .fastaq, .VCF, or .BAM files) can be provided to consumer/patients on a 500 GB hard-drive. Mutations in the A1AT gene could indicate pathogenesis associated with traditional A1AT deficiency (lung or liver disease), but could also indicate neurological or neuromuscular pathologies (either before or after symptom onset). For example, known variants in A1AT gene present among ALS patient samples collected and deposited in the ALS data browser can be used (ALSdb: alsdb.org).

Traditionally used for genetic forms of alpha-1 antitrypsin deficiency, augmentation therapy (also referred to as replacement therapy), in which a patient receives an intravenous infusion of alpha-1 antitrypsin, could also be administered to patients suffering from a functional deficiency of alpha-1 anti-trypsin activity. For example, patients with wild-type serpinA1 may suffer from insufficient (and therefore functionally deficient) amounts of serpinA1, such as in a neuroinflammatory state where neutrophil elastase and trypsin can be elevated. Functional deficiency can occur in addition to, and may be further exacerbated by, serpinA1 mutations which could cause the protein itself to function sub-optimally.

Drugs used for genetic forms of alpha-1 antitrypsin deficiency, and also applicable to treatment of functional serpinA1 deficiency, may include FDA approved drugs including but not limited to Prolastin-C® and Prolastin-C Liquid® from Grifols, Aralast NP™ from Takeda, Zemaira® from CSL Behring and Glassia® from Kamada Ltd. A product named Trypsone® from Grifols is available in Spain. Prolastin has been marketed since 1988 and has an excellent safety record. Aralast NP and Zemaira were introduced to the marketplace in 2003 and Glassia was introduced in 2010. Each was approved by demonstrating that they were comparable to Prolastin in their safety and in augmenting blood and lung alpha-1 anti-trypsin levels.

It can be desirable to dampen the hyperactivity of the proteases in the brain as a therapeutic treatment for ALS. For example, administration of a blood-brain-barrier permeable therapy that acts as a trypsin or neutrophil elastase inhibitor may slow protein degradation and in turn, decrease cell death. Alternatively, autophagy activators could lead to the regulated death of cells containing mutant serpinA1 aggregates, rather than other more potentially destructive forms of cell death such as necrosis.

### Amyotrophic Lateral Sclerosis

As used herein, “amyotrophic lateral sclerosis” or “ALS” includes the spectrum of neurodegenerative syndromes known under the names of Classical (Charcot's) ALS, Lou Gehrig's disease, motor neuron disease (MND), progressive bulbar palsy (PRP), progressive muscular atrophy (PMA), primary lateral sclerosis (PLS), bulbar onset ALS, spinal onset ALS and ALS with multi-system involvement (Wijesekera L C and Leigh P N. Amyotrophic lateral sclerosis. Orphanet Journal of Rare Disease 2009, 4:3). Types of ALS include sporadic ALS, familial ALS, Western Pacific ALS, Juvenile ALS, and Hirayama Disease.

Types of ALS include sporadic ALS, familial ALS, and variants, including Western Pacific ALS, Juvenile ALS, and Hirayama Disease. The present disclosure provides a method for treating ALS, including sporadic ALS, familial ALS, and variants, including Western Pacific ALS, Juvenile ALS, and Hirayama Disease.

Approximately 10% of cases of ALS are familial. The remaining 90% are sporadic and thought to be multifactorial, with both environmental and genetic components contributing to disease susceptibility. The genetics of both FALS and SALS is complex. About 20% of cases with autosomal dominant FALS and 2% of patients with SALS show mutations in the copper/zinc superoxide dismutase (SOD1) gene on chromosome 21. Mutations in the gene are thought to cause disease through a toxic gain of function rather than causing impairment of the antioxidant function of the SOD 1 enzyme. Although genes other than SOD1 have been associated with familial ALS, including alsin (ALS2), senataxin (ALS4) or Angiogenin, the genetic defect remains to be identified in the majority of cases.

Sporadic and familial ALS (SALS and FALS, respectively) are clinically and pathologically similar, suggesting a common pathogenesis. Both forms produce similar pathological hallmarks, including progressive muscle weakness, atrophy, and spasticity, each of which reflects the degeneration and death of upper and lower motor neurons. Denervation of the respiratory muscles and diaphragm is generally the fatal event. In current medical practice, the terms “bulbar onset ALS” and “spinal onset ALS” have replaced the terms PBP and Charcot's ALS. Approximately two thirds of patients with typical ALS have a spinal form of the disease (limb onset) and present symptoms related to focal muscle weakness and wasting, where the symptoms may start either distally or proximally in the upper and lower limbs. Gradually, spasticity may develop in the weakened atrophic limbs, affecting manual dexterity and gait. Patients with bulbar onset ALS usually present with dysarthria and dysphagia for solid or liquids, and limb symptoms can develop almost simultaneously with bulbar symptoms, and in the vast majority of cases will occur within 1-2 years. Paralysis is progressive and leads to death due to respiratory failure within 2-3 years for bulbar onset cases and 3-5 years for limb onset ALS cases.

Western Pacific ALS is a unique neurological disease initially identified among the Chamorro people of Guam and is characterized by a combination of symptoms including stooped posture, a blank expressionless face, dementia, slow shuffling movement, a resting tremor that stops upon deliberate action, slow movements, and muscle atrophy that results in muscles dipping down in the hand. Some patients have Parkinsonism features combined with dementia (Parkinsonism Dementia Complex, PDC). In still others, only dementia is observed. Neuropathologically, all clinical forms of the disease result in a specific feature, neurofibrillary tangles, found in the cortex and in the spinal cord. Because the disease has aspects that resemble amyotrophic lateral sclerosis (ALS), Parkinson's disease (PD) and Alzheimer's disease (AD), this disease is known as Western Pacific ALS or amyotrophic lateral sclerosis-Parkinsonism dementia complex of Guam (ALS-PDC) and is also known as Lytico-bodig.

Juvenile ALS is an adolescent motor neuron disease that is clinically indistinguishable from ALS. Onset is between ages of twelve and sixteen years and occurs below the age of 25 years.

In Hirayama Disease, there is localized atrophy of one arm associated with increased reflexes implicating the presence of upper and lower motor neuron damage. However, Hirayama Disease involves a problem at the junction between the cervical spine and the skull where there is pressure on the cervical spinal cord. In order to detect Hirayama Disease, an MRI of the neck needs to be performed in different positions including neck flexion and extension.

Other syndromes related to the ALS spectrum of disorders include progressive bulbar palsy (PBP), progressive muscular atrophy (PMA), primary lateral sclerosis (PLS) and ALS with multi-system involvement. The present disclosure provides a method for treating a syndrome related to the ALS spectrum of disorders, such as progressive bulbar palsy (PBP), progressive muscular atrophy (PMA), primary lateral sclerosis (PLS), or ALS with multi-system involvement.

Progressive bulbar palsy (PBP) is a motor neuron disease, in which the nerves supplying the bulbar muscles are attacked. PBP is characterized by the degeneration of motor neurons in the cerebral cortex, spinal cord, brain stem, and pyramidal tracts. Progressive bulbar palsy symptoms can include progressive difficulty with chewing, talking, and swallowing. Patients can also exhibit reduced gag reflexes, weak palatal movements, fasciculations, and weak movement of the facial muscles and tongue. In advanced cases of PBP, the patient may be unable to protrude their tongue or manipulate food in their mouth.

Progressive muscular atrophy (PMA, also Duchenne-Aran muscular atrophy or Duchenne-Aran disease) is a motor neuron disease which affects the lower motor neurons. Symptoms of PMA include atrophy, fasciculation, and muscle weakness. Some patients have symptoms restricted to the arms or legs.

Primary lateral sclerosis (PLS) is a neuromuscular disease characterized by progressive muscle weakness in the voluntary muscles. PLS affects upper motor neurons. Symptoms include difficulty with balance and weakness and stiffness in the legs. Other common symptoms are spasticity (involuntary muscle contraction due to the stretching of muscle). There may also be difficulty in breathing in the later stages of the disease, causing those patients to develop ventilatory failure. Hyperreflexia is another feature of PLS as seen in patients presenting with the Babinski's sign. Some people present with emotional lability and bladder urgency, and occasionally people with PLS experience mild cognitive changes detectable on neuropsychological testing, particularly on measures of executive function.

In some embodiments, the benefits of administering the compositions to alleviate the symptoms or treat ALS can be evaluated by the following tests: a reduction in the rate of decrease in the ALSFRS-R score, the Manual Muscle Testing (MMT) score, the Slow Vital Capacity (VC) percent predicted value, the ALS-Specific Quality of Life (ALSSQoL) score, and EuroQol-5 Dimensions (EQ-SD) Health Outcomes Scale score. Another suitable test for evaluation includes a reduction in the rate of increase in the Zarit Burden Interview (ZBI) score. Another suitable test for evaluation is the use of electrical impedance myography as a biomarker (Rutkove et al., Amyotroph Lateral Scler. 2012 September; 13(5):439-4). In some embodiments, the rate of decrease in, or the symptom measured by, the ALSFRS-R score is reduced by at least 15% as compared to an individual with ALS who is not administered an agent.

### Diagnosis and Symptoms of ALS

The methods described herein may also be useful for any one or more of the following: 1) preventing loss of skeletal muscle associated with amyotrophic lateral sclerosis; 2) treating muscle weakness associated with amyotrophic lateral sclerosis; 3) strengthening skeletal muscle in an individual having amyotrophic lateral sclerosis; 4) treatment of muscle wasting associated with amyotrophic lateral sclerosis; 5) treating dyspnea associated with muscle changes in amyotrophic lateral sclerosis; and 6) improving fatigue resistance of muscle in amyotrophic lateral sclerosis. Skeletal muscle includes, but is not limited to, gastrocnemius muscle, tibialis muscle, soleus muscle, and extensor digitorum longus (EDL) muscle, quadriceps, hamstrings, postural muscles, hand muscles, triceps, biceps, masseter and other jaw muscles, and intercostal and other respiratory muscles. The present application encompasses any of these methods.

In some embodiments, the individual has been diagnosed with or is suspected of having amyotrophic lateral sclerosis, through either conventional or unconventional means used to diagnose a patient with ALS. Unconventional means can include, but is not limited to, genotyping of serpinA1 sequence or quantification of serpinA1 protein abundance or activity in addition to other symptoms of disease. In some instances, the amount of serpinA1 in patients with ALS may fall within the physiological range (0.9-2.3 g/L), however, the anti-trypsin activity of the protein is lower than wild-type protein, e.g., in the case of hypofunctional serpinA1 variants falling within the physiological range of A1AT protein abundance (0.9-2.3 g/L). Therefore, serpinA1/A1At activity itself (instead of or in addition to A1AT protein abundance) can be compared to the activity in a reference sample having physiological amounts (between 0.9-2.3 g/L) of wild-type A1AT. Likewise, anti-trypsin activity can be determined in a reference sample generally considered to have a wild-type level of anti-trypsin activity, e.g., in a reference sample having physiological amounts (between 0.9-2.3 g/L) of wild-type A1AT. The amount and/or activity of A1AT can be determined in patient samples. The amount and/or activity of A1AT can be compared to a reference sample. The amount and/or activity of A1AT determined to be in a reference sample (e.g., a biological sample having physiological amounts of wild-type A1AT) can be used to establish a threshold. Activity and/or abundance values below the reference sample threshold indicate hypofunction and/or decreased amounts of A1AT. Neutrophil elastase activity can be determined as a proxy for A1AT function and/or abundance. An over-abundance of neutrophil elastase can indicate hypofunction of A1AT. Neutrophil elastase activity can be determined in a reference sample generally considered to have a wild-type level of activity, e.g., a biological sample having physiological amounts of neutrophil elastase and/or physiological amounts of wild-type A1AT. For example, neutrophil elastase protein level and/or activity above a threshold level, e.g., the level and/or activity correlating to a reference sample, can indicate hypofunction of A1AT in the biological sample. Reference samples can be taken from organs or tissue including, but not limited to, blood, cerebrospinal fluid, muscle, or a gastrointestinal tract. Levels and/or activity correlating to a reference sample can include those values known in the art, e.g., levels generally considered physiological.

Subjects and their respective biological samples can be characterized with respect to the levels and/or activities of proteins. Subjects and their respective biological samples can be characterized with respect to the levels and/or activities of proteins with respect to a reference sample, such as assessed to be above or below a threshold level and/or activity of a reference sample. Levels and/or activities include any of those of any protein described herein, such as serpinA1/A1AT, neutrophil elastase, serine proteases, and/or other proteases. For example, subjects can be characterized as having a deficiency in an alpha-1 antitrypsin activity when (1) a A1AT protein level below a threshold range of 0.9 and 2.3 g/L is detected in the biological sample, (2) an A1AT activity below a threshold activity correlating to activity of a reference sample comprising wild-type A1AT protein level in a range of 0.9 and 2.3 g/L is detected in the biological sample, and/or (3) an neutrophil elastase protein level above a threshold level correlating to neutrophil elastase protein levels in a reference sample comprising wild-type A1AT protein level in a range of 0.9 and 2.3 g/L is detected in the biological sample.

Therapies can be administered following characterization of a subject or their respective samples, such as characterized as having the deficiency in an alpha-1 antitrypsin activity and/or as having the excess of serine protease activity. Therapies can be administered following characterization of a subject with respect to levels and/or activities of proteins.

In some embodiments, the individual exhibits one or more symptoms associated with amyotrophic lateral sclerosis. In some embodiments, the individual is a human. In some embodiments, the individual is at least about any of 35, 40, 45, 50, 55, 60, 65, 70, 75, 80, or 85 years old. In some embodiments, the individual is a male. In some embodiments, the individual is a female. In some embodiments, the individual has been previously treated for amyotrophic lateral sclerosis. In some embodiments, the individual has not previously been treated for amyotrophic lateral sclerosis. Currently no treatment can reverse the damage of ALS. There are two approved medicines that slow progression of the disease, Riluzole (Rilutek), an oral treatment and Edaravone (Radicava), an intravenous infusion.

Amyotrophic lateral sclerosis covers a spectrum of neurodegenerative syndromes characterized by progressive muscular paralysis reflecting degeneration of motor neurons in the brain and spinal cord. It is a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

Amyotrophic lateral sclerosis can be diagnosed by observation of symptoms and signs. An individual may receive neurologic examinations at regular intervals to see whether symptoms are getting worse. Symptoms may include muscle weakness, muscle atrophy, hyperreflexia (overactive reflexes, including twitching and spastic movement), and spasticity (tightening and contraction of muscles, muscle stiffening). Tests can be run to obtain definitive information to diagnose ALS or rule out diseases other than ALS. Such tests include electromyography (EMG), nerve conduction velocity (NCV) test, magnetic resonance imaging (MRI), spinal tap, x-rays, myelogram of the cervical spine, and muscle and/or nerve biopsy.

In some embodiments, the individual has early stage amyotrophic lateral sclerosis. In some embodiments, the individual has middle stage amyotrophic lateral sclerosis. In some embodiments, the individual has late stage amyotrophic lateral sclerosis.

### Pharmaceutical Compositions and Methods of Treatments

The compositions described herein in some embodiments are present in pharmaceutical compositions. The pharmaceutical compositions may further comprise one or more pharmaceutically acceptable carrier (or excipients). A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. For example, neutrophil elastase inhibitor compound ursolic acid can be administered with a liposomal or nano-carrier mediated formulation (known as UANL) to improve the delivery of ursolic acid to human subjects, increasing its efficacy (Ramos-Hyrb et al, 2017). Aralast NP includes a human protein has been isolated from pooled human serum samples and lyophilized and reconstituted with albumin (less than or equal to 5 ml/mL), polyethylene glycol (less than or equal to 112 mcg/mL), polysorbate 80 (less than 50 mcg/mL), sodium (less than 230 micromol/mL), trit-n-butyl phosphate (less than 1.0 mcg/mL) and zinc (less than 3 mg/L). The concentration of functionally active alpha1-protease inhibitor in Aralast NP is greater than or equal to 16 mg/mL and the specific activity is 0.55 mg active alpha1-protease inhibitor/mg of total protein. In some embodiments, the pharmaceutical composition is sterile.

The pharmaceutical composition can be a nucleic acid packaged in a lipid nanoparticle that results in the expression of functional serpinA1. For example, Intellia's Crispr/Cas-9 gene-editing A1AT therapy restores alpha-1 anti-trypsin activity in the liver of non-human primates to physiological levels after a single dose. Gene-editing approaches that restore functional A1AT/serpinA1 activity can include restoring activity in cells including, but not limited to, hepatocytes, stem cells, myocytes, neurons, glia, and stem cells for the treatment for ALS.

The pharmaceutical composition can be an inhibitory nucleic acid (e.g., RNAi) that knocks down or disrupts the expression of a hypofunctional variant of the serpinA1 gene, such as the RNAi therapy (ARO-AAT) developed by Arrowhead Pharmaceuticals. RNAi approaches that reduce mutant A1AT/serpinA1 expression can include reducing expression in cells including, but not limited to, hepatocytes, stem cells, myocytes, neurons, glia, and stem cells could be an effective treatment for ALS. Inhibitory nucleic acids, include, but are not limited to, a dsRNA (e.g., an siRNA or shRNA), a clustered regularly interspaced short palindromic repeats (CRISPR), a transcription-activator like effector nuclease (TALEN), or a zinc finger endonuclease (ZFN). SerpinA1 genes can be genetically modified within a cell, e.g., having a genome editing using CRISPR, TALEN, or ZFN-based approaches. SerpinA1 expression can be transiently reduced, such as through RNAi-based approaches (e.g., an siRNA or shRNA).

Also provided here are unit dosage forms comprising pharmaceutical compositions described herein. These unit dosage forms can be stored in a suitable packaging in single or multiple unit dosages and may also be further sterilized and sealed. Unit dosage forms can be provided, for example, in the form of tablets, capsules, vials, and any other forms described herein.

In some embodiments, there is provided a composition (such as a pharmaceutical composition, for example a unit dosage) comprising an agent as described herein, or a pharmaceutically acceptable salt thereof, wherein the amount of the agent in the composition (such as pharmaceutical composition) is included in any of the following ranges: about 5 to about 10 mg, about 10 to about 20 mg, about 20 to about 30 mg, about 30 to about 40 mg, about 40 to about 50 mg, about 50 to about 60 mg, about 60 to about 70 mg, about 70 to about 80 mg, about 80 to about 90 mg, about 90 to about 100 mg, about 100 to about 110 mg, about 110 to about 120 mg, about 120 to about 130 mg, about 130 to about 140 mg, about 140 to about 150 mg, about 150 to about 160 mg. In some embodiments, the amount of the agent in the composition is about 20 to about 160 mg, including for example about 50 to about 150 mg, 80 to about 150 mg, about 90 to about 140 mg, about 100 to about 120 mg. In some embodiments, the composition is suitable for oral administration.

In some embodiments, the composition is provided in a slow release form.

Also provided are articles of manufacture comprising the compositions, formulations, and unit dosages described herein in suitable packaging for use in the methods of treatment, methods of administration, and dosage regimens described herein. Suitable packaging for compositions described herein are known in the art, and include, for example, vial (such as sealed vials), vessels (such as sealed vessels), ampules, bottles, jars, flexible packaging (e.g., sealed Mylar or plastic bags), and the like. These articles of manufacture may further be sterilized and/or sealed.

### Dosages and Administration Route

The dosage of the compositions described herein administered to an individual (such as a human) may vary with the particular composition, the method of administration, and the particular stage of amyotrophic lateral sclerosis. The amount should be sufficient to produce a desirable response, such as a therapeutic or prophylactic response against amyotrophic lateral sclerosis. In some embodiments, the amount of the composition is a therapeutically effective amount. In some embodiments, that amount of the composition is a prophylactically effective amount. In some embodiments, the amount of total the agent in the composition is below the level that induces a toxicological effect (i.e., an effect above a clinically acceptable level of toxicity) or is at a level where a potential side effect can be controlled or tolerated when the composition is administered to the individual. Methods of administration of A1AT replacement therapies and/or other agents that modify SerpinA1 biology described herein are generally known to those of skill in the art. For example, Aralast NP is a sterile, lyophilized preparation of human alpha-1 antitrypsin protein derived from large pools of human plasma. Aralast NP contains approximately 2% Alpha-1 protease inhibitor with C-terminal truncation of lysine 394. 60 mg/kg body weight of Aralast NP is administered weekly via intravenous infusion. In another example, Sivelestat, a neutrophil elastase inhibitor is administered at 0.2-0.4 mg/kg/h via continuous intravenous infusion for 14 days. In another example, ursolic acid, isolated from the rosemary herb, can be administered at 450 mg/day orally.

In some embodiments, the amount of an agent or a pharmaceutically acceptable salt thereof in the composition is included in any of the following ranges: about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg. In some embodiments, the amount of a pharmaceutical composition includes at least about any of 0.1 mg/kg, 0.5 mg/kg, 1 mg/kg, 2.5 mg/kg, 5 mg/kg, 7.5 mg/kg, 10 mg/kg, 15 mg/kg, or 20 mg/kg or the composition includes less than about any of 35 mg/kg, 30 mg/kg, 25 mg/kg, 20 mg/kg, 15 mg/kg, 10 mg/kg, 5 mg/kg, 2.5 mg/kg, 2 mg/kg, 1 mg/kg, 0.5 mg/kg, or 0.1 mg/kg.

Exemplary dosing frequencies include, but are not limited to, weekly without break; weekly, three out of four weeks; once every three weeks; once every two weeks; weekly, two out of three weeks. In some embodiments, the composition is administered about once every 2 weeks, once every 3 weeks, once every 4 weeks, once every 6 weeks, or once every 8 weeks. In some embodiments, the composition is administered at least about any of once, twice, three times, four times, five times, six times, or seven times (i.e., daily) a week. In some embodiments, the intervals between each administration are less than about any of 6 months, 3 months, 1 month, 20 days, 15, days, 12 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week. In some embodiments, the composition is administered daily. In some embodiments, the composition is administered twice daily. In some embodiments, the composition is administered at least once (such as at least any of 2×, 3×, or 4×) daily.

The administration of the composition can be extended over an extended period of time, such as from about a month up to about seven years or life-long. In some embodiments, the composition is administered over a period of at least about any of 2, 3, 4, 5, 6, 7, 8, 9, 10, 11, 12, 18, 24, 30, 36, 48, 60, 72, or 84 months or life-long. In some embodiments, the composition is administered over a period of at least one month, wherein the interval between each administration is no more than about a week.

The compositions described herein can be administered to an individual (such as human) via various routes, including, for example, intravenous, intra-arterial, intraperitoneal, intraportal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used.

Methods of administering antibody therapies described herein are generally known to those of skill in the art. Pharmaceutical compositions that include antibodies are preferably suitable for parenteral administration to a human (e.g., according to the standard of the FDA). Pharmaceutical compositions for parenteral administration include sterile formulations that are substantially isotonic and manufactured under GMP conditions. Pharmaceutical compositions that include antibodies can be provided in unit dosage form (i.e., the dosage for a single administration). Pharmaceutical compositions that include antibodies can be formulated using one or more pharmaceutically acceptable carriers, diluents, excipients or auxiliaries. Pharmaceutically acceptable means suitable for human administration, e.g., approved or approvable by the FDA. The formulation for administration of antibodies depends on the route of administration chosen. For injection, antibodies can be formulated in aqueous solutions, such as physiologically compatible buffers including, but not limited to physiological saline or acetate buffer (to reduce discomfort at the site of injection), Ringer's solution, or Hank's solution. The solution for administration of antibodies can contain formulatory agents such as suspending, stabilizing and/or dispersing agents. Alternatively antibodies can be in lyophilized form for constitution with a suitable vehicle, e.g., sterile pyrogen-free water, before use.

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Pharmaceutical Formulations and Administration

The pharmaceutical compositions described herein may be formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the embodiments may be administered by a suitable route of delivery, such as oral, parenteral, rectal, nasal, topical, or ocular routes, or by inhalation. Preferably, the compositions are formulated for intravenous or oral administration.

For oral administration, the compositions may be provided in a solid form, such as a tablet or capsule, or as a solution, emulsion, or suspension. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating. The oral formulations may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Capsules containing ursolic acid, for example, can contain maltodextrin, gelatin, silica, magnesium stearate, titanium dioxide, and sodium copper chlorophyllin.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg povidone, gelatin, hydroxypropylmethyl cellulose), lubricant, inert diluent, preservative, disintegrant (eg sodium starch glycolate, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents. The human alpha1-protease inhibitor protein Aralast NP consists of lyophilized protein that is reconstituted with additional compounds in liquid for intravenous infusion.

For parenteral use, including intravenous, intramuscular, intraperitoneal, intranasal, or subcutaneous routes, the compositions may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Formulations suitable for parenteral including intravenous administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or art appropriate fraction thereof, of an active ingredient.

### Drug Combinations

The methods of the embodiments comprise administering an effective amount of at least one compound of the embodiments; optionally the compound may be administered in combination with one or more additional therapeutic agents, particularly therapeutic agents known to be useful for treating amyotrophic lateral sclerosis afflicting the subject.

The additional active ingredients may be administered in a separate pharmaceutical composition from a compound of the embodiments or may be included with a compound of the embodiments in a single pharmaceutical composition. The additional active ingredients may be administered simultaneously with, prior to, or after administration of a compound of the embodiments.

In certain embodiments, the additional therapeutic agent is selected from the group consisting of CK-2017357, olesoxime (TRO 19622), arimoclomol, riluzole, tretinoin and pioglitazone HCl, AVP-923, memantine, talampanel, tauroursodeoxycholic acid (TUDCA), thalidomide, olanzapine, carbamazepine, alvelestat, sivelestat, KNS-760704, lithium carbonate, NPOO1, ONO-2506PO, tamoxifen, creatine monohydrate, coenzyme Q10, YAM80, sodium phenylbutyrate, pyrimethamine, R(+)pramipexole dihydrochloride monohydrate, vitamin E, minocycline, topiramate, gabapentin, AEOL-10150, stem cell injections, SB-509, autologous bone marrow-derived stem cells, ceftriaxone, E0302 (mecobalamin), MCI-186, glatiramer acetate, insulin-like growth factor-1 (IGF-1), ISIS 333611, sNN0029, GSK1223249, brain-derived neurotrophic factor (BDNF), and anti-CD40L antibody.

### Kits

The present application also provides kits, medicines, compositions, and unit dosage forms for use in any of the methods described herein.

Kits provided herein include one or more containers comprising any one of the compositions described herein and/or other agent(s), and in some embodiments, further comprise instructions for use in accordance with any of the methods described herein. The kit may further comprise a description of selection of individual suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of the invention are in suitable packaging. Suitable packaging include, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the agent as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the pharmaceutical compositions and instructions for use and packaged in quantities sufficient for storage and use in pharmacies, for example, hospital pharmacies and compounding pharmacies.

Also provided are medicines, compositions, and unit dosage forms useful for the methods described herein. For example, the present disclosure provides, in some embodiments, the agent formulated for the manufacture of a medicament for treating amyotrophic lateral sclerosis in an individual having amyotrophic lateral sclerosis. The present disclosure provides, in some embodiments, the agent for the manufacture of a medicament for prolonging survival of an individual having amyotrophic lateral sclerosis. The present disclosure provides, in some embodiments, the agent for the manufacture of a medicament for delaying the development of amyotrophic lateral sclerosis in an individual having amyotrophic lateral sclerosis. The present disclosure provides, in some embodiments, the agent for the manufacture of a medicament for preventing lean mass loss of an individual having amyotrophic lateral sclerosis. The present disclosure provides, in some embodiments, the agent for the manufacture of a medicament for preventing muscle wasting of an individual having amyotrophic lateral sclerosis. The present disclosure provides, in some embodiments, the agent for the manufacture of a medicament for improving quality of life in an individual having amyotrophic lateral sclerosis.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2^{nd} Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Example 1

The invention features a method of treating ALS. A biological sample is obtained from a patient characterized as having muscle weakness based on grip strength measurements and weight. The biological sample is assessed for protease activity, such as trypsin or elastase levels, which increases under inflammatory conditions. If the sample reveals elevated trypsin or elastase activity, the patient is treated with administration of 450 mg of the protease inhibitor ursodiol acid or derivative thereof for eight (8) weeks (as in Bang et al, 2017). During treatment, trypsin or elastase levels, grip strength and weight are measured to assess the treatment and to see an increase in levels and performance.

### Example 2

In ALS mouse models, like mutant SOD mice (SOD1G93A)), ALS is diagnosed based on hanging wire grip test (where the latency of falling is measured in seconds), grip strength analysis, rotarod time trial test, where a decrease in time balanced on the rod correlates to disease positive mice. Weight is measured to determine hypotrophy of muscle mass over time. Low levels of serum serpinA1 proteins levels, serum protease inhibitor function, serum trypsin protein levels, and serum functional trypsin level correlates with ALS. Once the mice have been diagnosed with ALS, the mice are given daily treatments of ursolic acid (200 mg/kg) over the course of eight weeks. Levels of serum serpinA1 proteins, serum protease inhibitor function, serum trypsin protein, and serum functional trypsin are assessed over time monitoring for an increases. Grip test and rotarod times are assessed over time as a correlation for delay in disease progression. Overall, mice receiving treatments outperform the non-treatment group.

### Example 3: Testing for Functional Deficiency in Serine Protease Activity (to Determine if Deficits in A1AT Function Correlate with Pathology)

In another method of treatment, a biological fluid is collected from a patient. Examples of biological samples include blood and cerebrospinal fluid. The biological fluid is assessed to detect serine protease activity. Method of assessing serine protease activity include assay methodologies where protease activity cleaves a fluorescent reporter peptide, which loses its fluorescence upon cleavage. Decreased level of fluorescence is an indicator of protease activity within the sample.

When the sample exhibits elevated protease activity above the normal physiological range (e.g., the activity found in a reference sample having 0.9 and 2.3 g/L of wild-type A1AT), a therapeutic protease inhibitor is administered. Examples of therapeutic inhibitors include ursolic acid or a tripterpine or fragments thereof. Following the therapeutic treatment, a patient's functional protease activity is re-assessed. Patients treated with therapeutic inhibitors exhibit less functional protease activity when compared to the controls. Patient's symptoms, including muscle weakness and fatigue, ameliorate following treatment where overactive protease activity is diminished.

### Example 4: Testing for the Presence of a Hypofunctional Copy of a Serine Protease (to Determine if Ursolic Acid Treatment Might be Effective)

A biological fluid is collected from a patient. Examples of biological samples include blood and cerebrospinal fluid. DNA is extracted and sequenced using methods known in the art. The resulting sequence is compared to the serpinA1 protein. Identical matches that code for the 418 amino acid protein have wild-type serpinA1 gene resulting in normal serine protease levels. Samples matching the PiS allele snp, PiZ allele snp or having a premature stop codon result in hypofunctional serine protease activity. Nonmatching sequences are assessed to determine serine protease activity.

Biological samples are treated with recombinant trypsin and the level of trypsin inhibition is assessed. Samples with trypsin activity are treated with a trypsin inhibitor, ursolic acid or derivative thereof, to determine a therapeutic biologic to administer in human and mouse subjects.

### Example 5: Assessment of A1AT Abundance in Blood (to Determine if Ursolic Acid Treatment Might be Effective)

A biological fluid is collected from a patient. Examples of biological samples include blood and cerebrospinal fluid. Trypsin activity is assessed using methods known in the art. The normal amount of trypsin activity can be measured for a reference sample, i.e. a biological fluid containing wild-type proteases (e.g., the activity found in a reference sample having 0.9 and 2.3 g/L of wild-type A1AT). Samples with trypsin activity and/or A1AT levels above or below this range are characterized as having hyper-A1AT levels or hypo-A1AT levels, respectively. Protease inhibitors, including ursolic acid, a triterpene compound or derivatives thereof are administered to subjects with hypo-serpinA1 and hyper-serpinA1 levels. Following administration, trypsin activity is reassessed and a decrease in serpinA1 protein levels is observed.

### Example 6: Screening Subjects for Disrupted Protease Activity

The coding region of the serpinA1 gene is sequenced to determine if the subject has wild-type alleles or mutant alleles that may be hypofunctional or prone to aggregation. Methods of sequencing the serpinA1 gene include collecting blood from a patient, isolating cells, and extracting genomic DNA. DNA primers amplify the coding regions of the serpinA1 gene in polymerase chain reactions, and subsequently the amplicon is sequenced. Sequence analysis determines whether the wild-type allele is present or a mutant copy of the serpinA1 gene is present, including pathogenic variants which include but are not limited to the PiS (rs17580), PiZ (rs28929474), PiM also known as M1Ala/M1Val (rs6647), M3 (rs1303), and M2/M4 (rs709932) alleles (see Table 1).

TABLE 1
Variants of human serpinA1 allele.

| Sequence | Allele | | |
|---|---|---|---|
| ID No | abbreviation | Mutation | Variant |
| 1 | PiS | Glu264Val | rs17580 |
| 2 | PiZ | Glu342Lys | rs28929474 |
| 3 | PiM/M1A | Ala213 | rs6647 |
| 4 | PiM (Malton) | Null | rs775982338 |
| 5 | PiM (Heerlen) | Null | rs199422209 |
| 6 | PiM (Mineral | Null | rs28931568 |
| | Springs) | | |
| 7 | PiM (Procida) | Null | rs28935169 |
| 8 | PiNull (Granite | Null | rs267606950 |
| | Falls) | | |
| 9 | PiNull | Null | rs199422211 |
| | (Bellingham) | | |
| 10 | PiNull (Mattawa) | Null | rs28929473 |
| 11 | PiNull (Devon) | Null | rs11558261 |
| 12 | PiQ0 | Null | rs28931572 |
| | (Ludwigshafen) | | |
| 13 | PiS (Iiyama) | Null | rs55819880 |

Mutant A1AT may not function properly, resulting in a loss of serine protease inhibition. Alternatively, wild-type A1AT may not be expressed in sufficient quantities to counteract serine proteases. Either of these processes, or both in tandem, may result in tissue damage as a consequence of overactive protein degradation. Therefore, one method of assessing the function of A1AT is measuring the quantity of one of its targets, neutrophil elastase. An over-abundance of neutrophil elastase indicates hypofunction of A1AT.

Neutrophil elastase (NE) is quantified from a blood sample taken from a subject using the Sandwich-ELISA principle (Elabscience, E-EL-H1946). The microtiter ELISA plate provided in the kit has been pre-coated with an antibody specific to human NE. Standards or samples are added to the microtiter ELISA plate wells and combined with the specific antibody. Then a biotinylated detection antibody specific for human NE and Avidin-Horseradish Peroxidase (HRP) conjugate are added successively to each microtiter plate well and incubated. Free components are washed away. The substrate solution is added to each well. Only those wells that contain human NE, biotinylated detection antibody and Avidin-HRP conjugate will appear blue in color. The enzyme-substrate reaction is terminated by the addition of stop solution and the color turns yellow. The optical density (OD) is measured spectrophotometrically at a wavelength of 450 nm±2 nm. The OD value is proportional to the concentration of human NE. The concentration of human NE in the samples is calculated by comparing the OD of the samples to the standard curve. Samples from patients with ALS have higher levels of NE. Patients with elevated NE are candidates for drug treatment.

Normal A1AT is secreted from cells, but mutant forms are improperly processed and aggregate inside the cell.

A1AT protein is quantified from a blood sample taken from a subject. The reference range of normal concentrations of A1AT in blood is between 19.2-53.0 μM (99.9-275.4 mg/dL). Measurements below this value indicate a deficiency. The serum level of A1AT is determined by latex-enhanced immunoturbidimetric assay using Roche COBAS INTEGRA 400 plus analyzer (Roche Diagnostics, Indianapolis, USA), as in Ferrarotti et al, 2012 (Ferrarotti, Ilaria et al. “Serum levels and genotype distribution of al-antitrypsin in the general population” Thorax 2012 August; 67(8):669-74. doi: 10.1136/thoraxjnl-2011-201321).

A sandwich ELSA can also be used to assess the quantity of A1AT that is secreted from cells. Human A1AT present in the test sample (blood, serum) is captured by anti-human alpha-1-antitrypsin antibody that has been preadsorbed on the surface of microtiter wells using a human serpinA1 ELISA kit (Abcam, ab108799). After sample binding, unbound proteins and molecules are washed off, and a biotinylated detection antibody is added to the wells to bind to the captured A1AT. A streptavidin-conjugated horseradish peroxidase (SA-HRP) is then added to catalyze a colorimetric reaction with the chromogenic substrate TMB (3,3′,5,5′-tetramethylbenzidine). The colorimetric reaction produces a blue product, which turns yellow when the reaction is terminated by addition of dilute sulfuric acid. The absorbance of the yellow product at 450 nm is proportional to the amount of A1AT analyte present in the sample and a standard curve can be generated. The A1AT concentration in the test samples are then quantified by interpolating their absorbance from the standard curve generated in parallel with the samples. After factoring sample dilutions, the A1AT concentration in the original sample can finally be calculated. The reference range of normal concentrations of A1AT in blood is between 19.2-53.0 μM (99.9-275.4 mg/dL). Measurements below this value indicate a deficiency.

Patients with ALS have disrupted protease activity, which manifests itself as an overabundance NE, a deficiency in functional A1AT, or a combination of both, and indicates patients are suitable candidates for drug treatment.

### Example 7: In Vitro Screening of Drugs Reduces Inflammation, Induces Autophagy and Promotes Cell Viability

### Drug Treatments

Alvelestat (AZD9668), a human neutrophil elastase inhibitor is added to cells at a concentration of 20 μg/ml, in mice at a concentration of 1-10 mg/kg twice daily via oral administration, and in humans at a dose of 60 mg-240 mg, twice daily via oral administration.

Sivelestat, a competitive human neutrophil elastase inhibitor is added to cells at a concentration of 100 μg/mL, in mice at a concentration of 100 mg/kg in a volume of 100 μL intraperitoneally administered weekly, or in humans at 0.2 mg/kg/h via intravenous infusion for 3 days.

Carbamazepine, a sodium channel blocker and autophagy activator, is added to cells at a concentration of 100 μg/mL, dosed in mice at 200 mg/kg once daily, in humans at an increasing concentration starting at 400 mg/day, and increasing by 200 mg/day until 1200 mg/day is reached.

Ursolic acid, a naturally occurring neutrophil elastase inhibitor and autophagy activator, is added to cells at a final concentration of 15 uM, in mice at a concentration of 10 mg/kg via intraperitoneal injection administered weekly, and in humans at a concentration of 250 mg, via oral administration, twice daily.

Tripterin (celastrol), a pentacyclic triterpenoid mitophagy activator derived from the peeled root of the Thunder god vine (Tripterygium wilfordii) is added to cells at a concentration of 5 μM, in mice at a concentration of 3-6 mg/kg a day via intraperitoneal injection or mixed into food at 0.004%, and in humans at a concentration of 250 mg, via oral administration, twice daily.

Purified A1AT protein is added to cells at a concentration of 50 μg/ml, administered to mice at a concentration of 100 μg/mL, administered to mice at a concentration of 10 μg/kg, intraperitoneally administered daily, or to humans at 60 mg/kg administered once weekly by intravenous infusion.

### Cell Lines

The efficacy of drug treatments are tested on neural cells in culture. In vitro experiments include induced pluripotent stem cells, (IPSCs), induced motor neurons (iMNs) derived from patients with sporadic or familial ALS, and patient-derived glial cultures (astrocyte, oligodendrocyte and microglia), primary neural cells from ALS animal models, and immortalized cell lines.

A method of testing the efficacy of drug compounds is screening compounds on patient-derived cells. A skin sample containing fibroblasts is taken from a patient with ALS and differentiated into induced pluripotent stem cells then into induced motor neurons as in Shi et al, 2019. Fibroblasts are seeded into a T75 flask coated with Matrigel (Corning) and passaged for 14 days. Reprogramming is performed on 96-well plates coated with 0.1% gelatin and laminin. Transcription factors that induce motor neuron (iMN) differentiation and 5 mg/mL polybrene are added to 100 μL fibroblast medium into each well of the 96-well plate. On day 5, primary mouse cortical glial cells from P1 pups are added to transduced cultures in glia medium containing minimal essential medium (MEM) (Life Technologies), 10% donor equine serum (HyClone), 20% glucose (Sigma-Aldrich), and 1% penicillin/streptomycin. On day 6, cultures are switched to N3 medium containing DMEM/F12 (Life Technologies), 2% FBS, 1% penicillin/streptomycin, N2 and B27 supplements (Life Technologies), 7.5 μm RepSox (Sellect), and 10 ng/mL each of GDNF, BDNF, CNTF (R&D Systems). iMN cultures are maintained in N3 medium, changed every day. Cultured motor neurons from both ALS patients (ALS-iMNs) and healthy controls (iMNs) are maintained.

Drug efficacy is assessed by incubating the cells with varying concentrations of compounds present in the medium to generate a dose response curve. Cell survival, autophagy, and inflammation are monitored in cell-based assays.

ALS-iMNs are treated with compounds for 3 days. Cell viability is assessed by counting the number of cells in each well, assessing morphology, and applying a DNA intercalating dye (propidium iodine, ThermoFisher Scientific P3566) to live cells attached to a tissue culture plate. Exclusion of the dye indicates live cells, whereas cells that fluoresce red indicate dead cells. Total cells are counted, and red fluorescent cells (dead) are counted. A decrease in the fraction of red fluorescent ALS-iMNs indicates a protective effect of the drug treatment.

In separate experiments, iMNs are treated with compounds for 3 days, and autophagy, a form of cell-directed cell death is assessed with the use of a fluorescent reporter of p62 localization (ThermoFisher Premo Autophagy Sensor RFP-p62 kit, P36241). The red-fluorescent autophagy sensor reagent is incubated with live cells overnight. The RFP is assessed and imaged with a fluorescent microscopy. When autophagy is induced the p62 protein localizes to the autophagosomes and is subsequently degraded. Conversely, with the inhibition of autophagy, the p62 protein accumulates in the autophagosome. The number of p62 positive spots are counted for each condition. Under baseline conditions, autophagy is impaired in ALS patient-derived iMNs compared to healthy subject-derived iMNs. RFP-p62 protein accumulates more in ALS-iMNs. Following treatment of ALS patient-derived iMNs with an autophagy activator, more cells undergo autophagy and less intracellular RFP-p62 aggregation is observed. A decrease in the number p62 spots in drug treated ALS-iMNs compared to vehicle-treated indicates a protective effect of the treatment.

The inflammatory status of the cultured cells following drug treatment is assessed by cytokine profiling. A multiplexed ELISA assay that measures release of several cytokines (including but not limited to IL-1B, TNF-alpha, IL-6) in tissue cultured cells is performed according to manufacturer's instructions (FirePlex Human Discovery Cytokines—Immunoassay Panel 1, ab243551). Drug treatment achieves an anti-inflammatory effect, which results in a reduced concentration of inflammatory molecules in the ALS-iMN supernatant compared to vehicle-treated ALS-iMNs.

Neutrophil elastase, A1AT, LAMP2 (lysosomal marker), GFAP (astrocyte marker) and Tuj1 (neuronal marker), expression is quantified by immunofluorescence in fixed ALS-iMNs. Cells are fixed in 4% paraformaldehyde for 1 hour at 4 degrees C., permeabilized with PBS containing 0.1% Triton-X (PBS-T) overnight at 4 degrees C., blocked with serum in 0.1% PBS-T for 2 hours and incubated with primary antibodies at 4 degrees C. overnight. Cells are then washed with 0.1% PBS-T and incubated with Alexa-Fluor conjugated secondary antibodies (Life Technologies) in blocking buffer for 2 hours at room temperature. Cells are stained with DAPI (Life Technologies) and then mounted on slides with Vectashield (Vector Labs). Images are acquired on a Nikon confocal microscope. Neuronal area is determined in FIJI (NIH). The following antibodies are used:

Antibodies recognizing A1AT (Abcam ab922; undiluted) or neutrophil elastase (Abcam ab68672, 1:100), mouse anti-Tujl (EMD Millipore, catalog AB9354, 1:1000), mouse anti-LAMP2 (DSHB, catalog H4B4, 1:4000). Intracellular localization and aggregation of A1AT is assessed by observing co-localization of A1AT with proteasomes (PSMA1, Invitrogen JU66-24, 1:100) or lysosomes (LAMP2). The extent of drug treatment attenuating glial reactivity is assessed with GFAP immunostaining (rat anti-GFAP, Invitrogen #13-0300). A1AT conglomerated with neurofilament is detected, as in Chou et al, 1998.

### Example 8: In Vivo Screening of Drugs Reduces Inflammation and Promotes Survival in Mouse Models of ALS

### Mouse Models of ALS

Mouse models of motor neuron degeneration include but are not limited to the mSOD mouse with mutant superoxide dismutase gene (G93A), including, for example, strains #002726: B6SJL-Tg(SOD1*G93A)1/Gur/J or #004435: B6.Cg-Tg(SOD1*G93A)1/Gur/J, available from the Jackson Lab, a mouse model with C9orf72 G4C2 hexanucleotide repeat expansion, a mouse model of ALS that overexpresses TARDBP (TDP43) in postnatal neurons, including, for example, strain #012836: B6; SJL-Tg(Thyl-TARDBP)4Singh/J (Wils, Hans et al. “TDP-43 transgenic mice develop spastic paralysis and neuronal inclusions characteristic of ALS and frontotemporal lobar degeneration.” Proceedings of the National Academy of Sciences of the United States of America vol. 107,8 (2010): 3858-63. doi:10.1073/pnas.0912417107) or #010700: B6.Cg-Tg(Prnp-TARDP*A315T)95Balo/J, available from The Jackson Lab) (Wegorzewska, Iga et al. “TDP-43 mutant transgenic mice develop features of ALS and frontotemporal lobar degeneration.” Proceedings of the National Academy of Sciences of the United States of America vol. 106,44 (2009): 18809-14. doi:10.1073/pnas.0908767106; Hatzipetros, Theo et al. “A Quick Phenotypic Neurological Scoring System for Evaluating Disease Progression in the SOD1-G93A Mouse Model of ALS.” Journal of visualized experiments: JoVE, 104 53257. 6 Oct. 2015, doi:10.3791/53257). Viral induction of motor neuron disease, including, for example neurovirulent murine leukemia retroviruses such as FrCasE and Friend viruses (Cardona, Sandra M et al. “Astrocyte Infection Is Required for Retrovirus-Induced Spongiform Neurodegeneration Despite Suppressed Viral Protein Expression.” Frontiers in neuroscience vol. 13 1166. 29 Oct. 2019, doi:10.3389/fnins.2019.01166).

The mouse models of A1AT dysfunction deficiency, is traditionally used as a genetic model of emphysema, chronic obstructive pulmonary disease, or liver disease. However, the serpinA1 null mice with 5 serpinA1 paralogs knocked out (Borel, Florie et al. “Editing out five Serpinal paralogs to create a mouse model of genetic emphysema.” Proceedings of the National Academy of Sciences of the United States of America vol. 115,11 (2018): 2788-2793. doi:10.1073/pnas.1713689115) is herein used to model some aspects of amyotrophic lateral sclerosis.

### In Vivo Screening of Drug Compounds in a Mouse Model of ALS

A method of testing drug efficacy in vivo involves drug treatments in a mouse model of disease. Measures of weight, motor performance, muscle activity, neurological score, survival, inflammation and histopathology are performed. Treatment of an ALS mouse model, such as mSOD (G93A) mice with drug commences at 8 weeks of age (P56) which precedes observable motor impairments that occur at P105-P110 or death at P130. 64 litter-matched gender-balanced mice are used to assess disease onset, motor function and life span in the drug efficacy study. There are 32 animals in each cohort (drug and placebo). The body weight of all mice is measured every 4 days at the same time from the first drug administration until death.

The rotarod test is performed to assess the onset and progression of motor disability during the course of the disease. Animals are trained three times per week on a rod rotating at 14 rotations per minute (rpm) and then were tested weekly from ages 8 to 18 weeks, with an arbitrary maximum time of maintenance on the rotating rod of 180 seconds. Clinical disease onset is defined as the first week when animals were not able to hold onto the rotating rod for 180 seconds.

Muscle activity is assessed by measuring the compound motor action potential (CMAP) amplitude from the tibialis anterior muscle on a weekly basis. CMAP amplitude is recorded as in Mancuso et al (“Presymptomatic electrophysiological tests predict clinical onset and survival in SOD1G93A ALS mice”. Muscle Nerve. 2014 December; 50(6):943-9. doi: 10.1002/mus.24237) with a stimulating microneedle electrode. The recording microelectrode is placed subcutaneously in the first third of the distance between the knee and ankle. The amplitude of the maximal M waves are measured. Increased motor neuron and motor axon excitability occurs in mSOD (G93A) mice. All potentials are amplified and displayed on a digital oscilloscope (Tektronix 450S) by setting appropriate for amplitude measurement from the baseline to the maximal negative peak. During the tests the mouse body temperature is kept constant between 34 and 36 degrees with a thermostat-controlled heating pad. Drug treated mSOD (G93A) animals have a higher CMAP amplitude compared to vehicle-treated animals.

The neurological score, which includes various measures including a phenotypic observation of posture and hindlimb splay reflex that occurs when mice are picked up by their tail is recorded according to Hatzipetros et al (“A Quick Phenotypic Neurological Scoring System for Evaluating Disease Progression in the SOD1-G93A Mouse Model of ALS.” Journal of visualized experiments: JoVE, 104 53257. 6 Oct. 2015, doi:10.3791/53257). The mice receive a score from 0 to 4, with 0 being pre-symptomatic to 4 being paralyzed daily. Drug treatment delays the decrement of neurological score in mSOD (G93A) mice.

Enhanced survival is measured by the number of days that drug treatment extends life. The endpoint of the disease is reached when the mouse is unable to right itself within 30 seconds of being placed on its side. The Kaplan-Meier analysis of survival reveals differences between treatment groups. Drug-treated mSOD (G93A) mice survive longer than vehicle-treated mice.

Another cohort of mice is sacrificed at 120 days of age, and their tissues are subjected to Western blot analysis or histopathological staining analysis.

At 120 days of age, half the cohort is transcardially perfused with 4% paraformaldehyde in phosphate-buffered saline. The brain, lumbar segment of the spinal cord, and tibialis anterior muscle are removed, post-fixed for 24 hours and cryopreserved in 30% sucrose. Transverse 30 μm sections are cut on a cryotome (Leica) in the motor cortex of the brain and between L2 and L5 segments. For each region or segment, a series of 10 sections each are collected on slides (Superfrost Plus).

For histopathological analysis of neuronal physiology, one slide of each animal is rehydrated for 1 min and stained for 2 hours with an acidified solution of 3.1 mM Cresyl violet. Then slides are washed with distilled water for 1 min, dehydrated and mounted with DPX (Fluka). Spinal motor neurons are identified by their localization in the ventral horn of the stained spinal cord sections and counted following strict size and morphological criteria. Only motor neurons with diameters larger than 20 μm and with a polygonal shape and prominent nucleoli are counted. Then number of motor neurons present in both ventral horns is counted in 4 serial sections of the L4 and L5 segments. Drug-treated mSOD (G93A) mice have more intact motor neurons in the ventral horn than vehicle-treated mice.

Luxol fast blue myelin staining (0.1%, abcam ab150675) is performed on muscle tissue cryosections to quantify the number and size of myelinated nerve fibers. Luxol Fast Blue solution is incubated for 24 hour at room temperature, then rinsed in water. Stain is differentiated by dipping in lithium carbonate solution and alcohol solution. Sections are rinsed in water and incubated in Cresyl violet for 2-5 min. Sections are rinsed in water, dehydrated in absolute alcohol (100%), cleared and mounted. Fewer and thinner nerve fibers are found in muscle sections from untreated mSOD (G93A) mice compared to drug-treated mice.

Immunohistochemistry is performed on a separate slide-mounted cryosections. Glial reactivity is assessed by staining for the astrocyte marker, GFAP. Tissue damage as a result of enhanced protein degradation is observed by staining for neutrophil elastase. Motor neurons are counted in the motor cortex using the motor neuron specific marker of neurofilament (SMI-32 antibody, EMD Millipore NE1023). Drug-treated mSOD (G93A) mice exhibit less astrocyte reactivity, less neutrophil elastase expression, and more intact motor neurons in the motor cortex than vehicle-treated animals.

Rather than undergo transcardial perfusion, the other half of the mice in this cohort are anaesthetized with isoflurane and decapitated. Blood is collected for cytokine profiling in the multiplexed ELISA kit (FirePlex Human Discovery Cytokines—Immunoassay Panel 1, ab243551). Samples from drug-treated mSOD (G93A) mice have a lower concentration of inflammatory cytokines than vehicle-treated animals.

The brain and spinal cord are removed and homogenized for Western blot. Total protein concentration of the protein lysates is measured using Pierce BCA Protein Assay Kit (ThermoFisher, catalog #23225). Fifty μg protein samples are prepared, run on a protein gel via electrophoresis, and transferred to a membrane for Western blotting. The membranes are blocked in 5% milkfat for 1 hour at room temperature, then incubated with 1:100 anti-NE antibody (Abcam ab68672) in 5% milkfat for 16-18 hours at 4 degrees C. or 2 hours at room temperature. After incubation, the membranes are washed and incubated with 1:10,000 secondary antibodies (goat anti-rabbit HRP-conjugated, BioRad, Catalong #) for 1 hour at room temperature. The membranes are incubated with Clarity Western ECL solution (BioRad, catalog #1705061) and then visualized with the ChemiDoc Touch auto-exposure setting. Lower levels neutrophil elastase are found in drug-treated compared to vehicle-treated animals.

### Example 9: Immunohistochemical Analysis of A1AT Expression in Neural Cells/Biochemical Analysis of A1AT Expression in Human Serum

Aggregation of misfolded A1AT is semi-quantitatively assessed by centrifugal separation and assay by Western blot of soluble (monomeric) and insoluble (polymeric) components, as described in Blomenkamp and Teckman (Blomenkamp Keith and Teckman Jeffrey. Semiquantitation of Monomer and Polymer Alpha-1 Antitrypsin by Centrifugal Separation and Assay by Western Blot of Soluble and Insoluble Components. Methods Mol Biol. 2017; 1639:227-234. doi: 10.1007/978-1-4939-7163-3_23). A tissue sample is obtained (brain, muscle, blood, liver) and homogenized on ice. Protein concentration is determined and the volume containing 5 μg of total protein is assayed. The sample is centrifuged at 15,000×g for 30 min at 4 degrees Celsius. The supernatant containing the soluble fraction is removed, leaving a pellet containing the insoluble fraction. Sample buffer is added to each tube and vortexed. The tubes are boiled for 5 min, cooled, and centrifuged. Samples are loaded onto a gel and run at 120V with an 85 mA current. The proteins are transferred at 100V at 250 mA for 1.5 hours. The membrane is blocked with 5% milkfat, then incubated with an HRP-conjugated polyclonal goat primary antibody against human A1AT (Abcam ab191350) for 25 min. The blot is rinsed and developed with ECLplus Detection Reagent, then signal is detected with a GE Typhoon 9410 laser scanner. The presence of insoluble aggregates of A1AT indicates that the subject is a suitable candidate for drug treatment. Drug treatment reduces A1AT aggregation in patient samples.

Expression of neutrophil elastase protein is also quantified by Western blot analysis from human plasma samples using a polyclonal rabbit anti-neutrophil elastase antibody (Abcam ab68672) at a concentration of 1 μg/ml. A significant increase in the abundance of neutrophil elastase indicates that a suitable subject is a candidate for drug treatment. Drug treatment reduces the abundance of neutrophil elastase in patient samples.

Histological analysis of tissue samples from subjects suspected of having a functional A1AT deficiency reveals protein aggregates that are identified by immunohistochemical analysis of tissue samples using Periodic-acid-Schiff-staining (PAS) indicating diastase-resistant globules. Tissue samples are fixed in 4% formaldehyde and sectioned at 30 μm thickness, and the reaction is performed according to the system protocol (395B-1KT-Sigma-Aldrich). Aggregated A1AT is a biomarker for ALS, and indicates that a subject is a suitable candidate for drug treatment. Drug treatment reduces A1AT aggregation in patient tissue.

Immunohistochemistry is performed to detect protein expression and localization of A1AT and neutrophil elastase. Tissue samples are fixed in 4% formaldehyde and sectioned at 30 μm thickness.

Antibodies recognizing A1AT (Abcam ab922; undiluted) or neutrophil elastase (Abcam ab68672; 1:100 dilution) are incubated in PBS with Triton-X overnight. Sections are washed and fluorescent secondary antibodies recognizing rabbit primary antibodies (Invitrogen AlexaFluor plus 488, A32731) are incubated at 1:1000 for 1 hour, and imaged with a fluorescent microscope. Subjects with a significant decrease in A1AT or an increase in neutrophil elastase compared to healthy controls are suitable candidates for drug treatment. Drug treatment increases functional A1AT expression or reduces the expression of neutrophil elastase in patient samples.

### Example 10: Combination Treatment Rescuing Protease Activity and Promoting Autophagy Enhances Neural Cell Survival In Vitro and In Vivo

The mutant protein contributes to pathology via both gain-of-function (aggregation) and loss-of-function mechanisms (hypofunction of anti-trypsin activity). The present disclosure provides formulations or compositions suitable for the inhibition of neutrophil elastase or autophagy activators. The combination therapies of the present disclosure may be administered alone or with another therapeutic.

A combination treatment treats both issues in tandem. For example:
1. An autophagy activator (such as ursolic acid, carbamazepine, or triterpene derived compound) in combination with a neutrophil elastase inhibitor (such as Alvelestat or Sivelestat)
2. Or an autophagy activator (such as ursolic acid, carbamazepine, or triterpene derived compound) in combination with an infusion of alpha-1-antitrypsin, such as Aralast.
3. Or an autophagy activator (such as ursolic acid, carbamazepine, or triterpene derived compound) in combination with a gene therapy that delivers serpinA1.
4. Or a neutrophil elastase inhibitor (such as Alvelestat or Sivlelestat) in combination with a gene therapy that delivers serpinA1.

Combination treatments improve patient survival better than monotherapy.

### Example 11: Treatment of Protease Dysfunction in a Clinical Trial of ALS Patients

A method of assessing drug efficacy involves measuring function according to the ALS-FRS (ALS-Functional Rating Scale), muscle activity, muscle strength, and weight in ALS patients in a double blind, placebo-controlled, multi-center, 16-week clinical trial of the drug.

Eligible humans patients are those diagnosed with ALS who either have a mutant serpinA1 allele or elevated serum neutrophil elastase levels are accepted into a clinical trial. The coding region of the serpinA1gene is sequenced. ALS patients with a mutant serpinA1 allele are included in the trial. In patients with wild-type copies of the serpinA1 gene, the level of neutrophil elastase in the serum is assessed. Patients with elevated NE compared to healthy controls are included in the trial. Other populations of ALS patients who may be included in the trial are ALS patients with a history of lung or liver dysfunction, COPD, or emphysema, ALS patients with a history of high cholesterol, ALS patients that don't carry any of the genetic mutations commonly associated with the disease (and thus the disease remains idiopathic).

Patients are dosed with the drug treatment for 16 weeks. Clinical efficacy is assessed at regularly scheduled study visits, including functional tests such as the ALS-FRS score. Primary outcome measures include incidence of adverse events, change in ALS-FRS score (time frame: approximately 16 weeks), and change in concentration of blood markers of neutrophil elastase. Secondary outcome measures include grip strength, inflammatory markers in blood, CMAP amplitude and maintenance of body weight.

At the start of the trial, the following data is collected: genetic sequence of serpinA1 gene, level of NE in serum, ALS-FRS (ALS Functional Rating Scale) score, serum cytokine concentrations, CMAP amplitude, grip strength, and weight.

Safety is assessed through the collection of adverse events (AEs), vital signs and physical examinations throughout the study. Blood samples are taken periodically throughout the study to assess pharmacokinetics of the drugs.

### Amyotrophic Lateral Sclerosis-Functional Rating Scale Score

One primary outcome measure is the monthly record of the ALS-FRS score. The ALS-FRS consists of 10 questions that ask the physician to rate his/her impression of the patient's level of functional impairment in performing one of ten common tasks, for example, climbing stairs. Each task is rated on a five-point scale, from 0=patient can't do it to 4=normal ability. Individual item scores are summed to produce a repeated score of between 0=worst and 40=best. ALS patients receiving the treatment have a slower progression of functional impairment. Another primary outcome measure is the concentration of blood biomarkers of neutrophil elastase activity. ALS patients receiving drug treatment have a significant decrease in serum neutrophil elastase.

A secondary outcome measure is grip strength. ALS patients receiving drug treatment lost grip strength at a slower rate than the untreated patients. Another secondary outcome measure is CMAP amplitude. Muscle strength decreases at a slower rate in drug treated ALS patients compared to untreated ALS patients. Another secondary outcome measure is body weight. ALS patients receiving drug treatment are expected to maintain baseline weight better compared to the untreated patients.

### Example 12: ALS Mutation Biomarkers

Whole genome sequencing is performed by commercial vendors from saliva samples provided by consumers or patients. Screening of the genome for genetic mutations known to cause or correlated with neurological diseases are also provided as a service. Mutations in the serpinA1 sequence are reported. Alternatively, primers within the serpinA1 gene are used to detect single-nucleotide polymorphisms.

### Sequences

### WT (SEQ ID NO: 14)

mpssyswgilllaglcclvpvslaedpqgdaaqktdtshhdqdhptfnkitpnlaefafslyrqlahqsnstniffspvsiatafamlslgtka dthdeileglnfniteipeaqihegfqellrtlnqpdsqlqlttgnglflseglklvdkfledvkklyhseaftvnfgdteeakkqindyvekgtq gkivdlykeldrdtvfalvnyiffkgkwerpfevkdteeedfhvdqvttvkvpmmkrlgmfniqhckklsswvllmkylgnataifflpd egklqhlenelthdiitkflenedrrsaslhlpklsitgtydlksvlgqlgitkvfsngadlsgvteeaplklskavhkavltidekgteaagamfl eaipmsippevkfnkpfvflmieqntksplfmgkvvnptqk
S allele (snp=17580): Glu288Val (SEQ ID NO: 15)
mpssyswgilllaglcclvpvslaedpqgdaaqktdtshhdqdhptfnkitpnlaefafslyrqlahqsnstniffspvsiatafamlslgtka dthdeileglnfniteipeaqihegfqellrtlnqpdsqlqlttgnglflseglklvdkfledvkklyhseaftvnfgdteeakkqindyvekgtq gkivdlykeldrdtvfalvnyiffkgkwerpfevkdteeedfhvdqvttvkvpmmkrlgmfniqhckklsswvllmkylgnataifflpd egklqhlvnelthdiitkflenedrrsaslhlpklsitgtydlksvlgqlgitkvfsngadlsgvteeaplklskavhkavltidekgteaagamfl eaipmsippevkfnkpfvflmieqntksplfmgkvvnptqk
Z allele (rs289294474): Glu366Lys (SEQ ID NO: 16)
mpssyswgilllaglcclvpvslaedpqgdaaqktdtshhdqdhptfnkitpnlaefafslyrqlahqsnstniffspvsiatafamlslgtka dthdeileglnfniteipeaqihegfqellrtlnqpdsqlqlttgnglflseglklvdkfledvkklyhseaftvnfgdteeakkqindyvekgtq gkivdlykeldrdtvfalvnyiffkgkwerpfevkdteeedfhvdqvttvkvpmmkrlgmfniqhckklsswvllmkylgnataifflpd egklqhlenelthdiitkflenedrrsaslhlpklsitgtydlksvlgqlgitkvfsngadlsgvteeaplklskavhkavltidkkgteaagamfl eaipmsippevkfnkpfvflmieqntksplfmgkvvnptqk

### REFERENCES

Bang, Hyun Seok et al. “Ursolic acid-induced elevation in serum irisin augments muscle strength during resistance training in men.” Korean J Physiol Pharmacol 18 (5): 441-6 (2014). doi: 10.4196/kjpp.2014.18.5.441.
Blomenkamp Keith and Teckman Jeffrey. Semiquantitation of Monomer and Polymer Alpha-1 Antitrypsin by Centrifugal Separation and Assay by Western Blot of Soluble and Insoluble Components. Methods Mol Biol. 2017; 1639:227-234. doi: 10.1007/978-1-4939-7163-3_23.
Borel, Florie et al. “Editing out five Serpinal paralogs to create a mouse model of genetic emphysema.” Proceedings of the National Academy of Sciences of the United States of America vol. 115,11 (2018): 2788-2793. doi:10.1073/pnas.1713689115
Capri, Miriam et al. “Recovery from 6-month spaceflight at the International Space Station: muscle-related stress in a proinflammatory setting.” FASEB J 33 (4): 5168-5180 (2019). doi: 10.1096/fj.201801625R.
Cardona, Sandra M et al. “Astrocyte Infection Is Required for Retrovirus-Induced Spongiform Neurodegeneration Despite Suppressed Viral Protein Expression.” Frontiers in neuroscience vol. 13 1166. 29 Oct. 2019, doi:10.3389/fnins.2019.01166
Chou, Samuel et al. “Serpin=serine protease-like complexes within neurofilament conglomerates of motoneurons in amyotrophic lateral sclerosis.” J Neurol Sci. 160 Suppl 1:S73-9 Oct. 1998, doi: 10.1016/s0022-510×(98)00202-0
Ebbert, Mark T W et al. “Conserved DNA methylation combined with differential frontal cortex and cerebellar expression distinguishes C9orf72-associated and sporadic ALS, and implicates SERPINA1 in disease.” Acta neuropathologica vol. 134,5 (2017): 715-728. doi:10.1007/s00401-017-1760-4
Ferrarotti, Ilaria et al. “Serum levels and genotype distribution of α₁-antitrypsin in the general population” Thorax 2012 August; 67(8):669-74. doi: 10.1136/thoraxjnl-2011-201321.
Halbgebauer, Steffen et al. “Modified serpinA1 as risk marker for Parkinson's disease dementia: Analysis of baseline data.” Scientific reports vol. 6 26145. 17 May. 2016, doi:10.1038/srep26145
Hatzipetros, Theo et al. “A Quick Phenotypic Neurological Scoring System for Evaluating Disease Progression in the SOD1-G93A Mouse Model of ALS.” Journal of visualized experiments: JoVE, 104 53257. 6 Oct. 2015, doi:10.3791/53257
Marciniak, Stefan et al. “New concepts in alpha-1 antitrypsin deficiency disease mechanisms”. Ann Am Thorac Soc 13. Suppl 4:S289-96 (2016). doi: 10.1513/AnnalsATS.201506-358 KV.
Mancuso, Renzo et al. “Presymptomatic electrophysiological tests predict clinical onset and survival in SOD1^{G93A} ALS mice”. Muscle Nerve. 2014 December; 50(6):943-9. doi: 10.1002/mus.24237.
Miranda, Elena et al. “A novel monoclonal antibody to characterize pathogenic polymers in liver disease associated with al-anti-trypsin deficiency. Hepatology vol. 2, Issue 3. 1078-1088 (2010). doi: 10.1002/hep.23760
Ramos-Hyrb, Ana et al. “Therapeutic potential of ursolic acid to manage neurodegenerative and psychiatric diseases.” CNS Drugs 31, 1029-1041 (2017). doi: 10.1007/s40263-017-0474-4.
Rutkove, Seward B et al. “Electrical impedance myography as a biomarker to assess ALS progression.” Amyotrophic lateral sclerosis: official publication of the World Federation of Neurology Research Group on Motor Neuron Diseases vol. 13,5 (2012): 439-45. doi:10.3109/17482968.2012.688837
Wallmark, A et al. “Monoclonal antibody specific for the mutant PiZ alpha 1-antitrypsin and its application in an ELISA procedure for identification of PiZ gene carriers”. PNAS 81 (18):5690-3 (1984). doi: 10.1072/pnas.81.18.5690.
Wegorzewska, Iga et al. “TDP-43 mutant transgenic mice develop features of ALS and frontotemporal lobar degeneration.” Proceedings of the National Academy of Sciences of the United States of America vol. 106,44 (2009): 18809-14. doi:10.1073/pnas.0908767106
Wijesekera, Lokesh C, and P Nigel Leigh. “Amyotrophic lateral sclerosis.” Orphanet journal of rare diseases vol. 4 3. 3 Feb. 2009, doi:10.1186/1750-1172-4-3
Wormser, Uri et al. “Reduced levels of alpha-1-antitrypsin in cerebrospinal fluid of amyotrophic lateral sclerosis patients: a novel approach for a potential treatment.” Journal of neuroinflammation vol. 13,1 131. 1 Jun. 2016, doi:10.1186/s12974-016-0589-4
Wils, Hans et al. “TDP-43 transgenic mice develop spastic paralysis and neuronal inclusions characteristic of ALS and frontotemporal lobar degeneration.” Proceedings of the National Academy of Sciences of the United States of America vol. 107,8 (2010): 3858-63. doi:10.1073/pnas.0912417107

All references, issued patents and patent applications cited within the body of the specification are hereby incorporated by reference in their entirety, for all purposes.

1. A method of treating amyotrophic lateral sclerosis (ALS) in a human subject in need thereof comprising administering to the subject an agent in an amount effective to i) increase an alpha-1 anti-trypsin (A1AT) activity and/or ii) decrease a serine protease activity in a target cell, tissue or organ of the subject, wherein the subject is characterized as having insufficient alpha-1-anti-trypsin activity in the target cell, tissue or organ.

2. A pharmaceutical composition comprising a) an agent in an amount effective to i) increase an alpha-1 anti-trypsin (A1AT) activity and/or ii) decrease a serine protease activity in a target cell, tissue or organ of a subject, wherein the subject is characterized as having insufficient alpha-1-anti-trypsin activity in the target cell, tissue or organ and b) a pharmaceutically acceptable carrier.

3. The pharmaceutical composition of claim 2, wherein the human or animal subject is characterized as having ALS or at risk of developing ALS.

4. The method of claim 1, wherein:
(I) the method comprises:
a. obtaining or having obtained a biological sample selected from cerebrospinal fluid, blood, urine, gastrointestinal or fecal matter, saliva, tears or tissue from the subject;
b. assessing or having assessed the biological sample for serpinA1/A1AT levels and/or alpha-1 antitrypsin activity, wherein the activity comprises serine protease inhibitor activity;
c. characterizing from the assessment whether the subject has a deficiency in an alpha-1 antitrypsin activity, optionally wherein the subject is characterized as having the deficiency in an alpha-1 antitrypsin activity when (1) a A1AT protein level below a threshold range of 0.9 and 2.3 g/L is detected in the biological sample, (2) an A1AT activity below a threshold activity correlating to activity of a reference sample comprising wild-type A1AT protein level in a range of 0.9 and 2.3 g/L is detected in the biological sample, and/or (3) an neutrophil elastase protein level above a threshold level correlating to neutrophil elastase protein levels in a reference sample comprising wild-type A1AT protein level in a range of 0.9 and 2.3 g/L is detected in the biological sample; and
d. administering to the subject an agent that inhibits serine protease activity in an amount effective to treat ALS when the subject is characterized as having the deficiency in an alpha-1 antitrypsin activity; or
(II) the method comprises:
a. obtaining or having obtained a biological sample selected from cerebrospinal fluid, blood, urine, gastrointestinal or fecal matter, saliva, tears or tissue from the subject;
b. assessing or having assessed the biological sample for serine protease activity, optionally wherein the subject is characterized as having a deficiency in an alpha-1 antitrypsin (serpinA1) activity, wherein the activity comprises serine protease inhibitor activity;
c. characterizing from the assessment whether the subject has an excess of serine protease activity, optionally wherein the subject is characterized as having the excess serine protease activity when serine protease activity above a threshold activity correlating to serine protease activity in a reference sample comprising wild-type A1AT protein level in a range of 0.9 and 2.3 g/L is detected in the biological sample; and
d. administering to the subject an effective dose of an agent that increases A1AT expression or activity in an amount effective to treat ALS when the subject is characterized as having the excess of serine protease activity.

5. (canceled)

6. The method of claim 4, wherein the characterization of the activity is with reference to a reference level in an organ or tissue, optionally wherein the organ or tissue is blood, cerebrospinal fluid, muscle, or gastrointestinal tract.

7. (canceled)

8. The method of any one of claim 4, wherein the symptom is stumbling; slurred speech; difficulty swallowing; muscle cramps; worsening posture; difficulty with holding the head up; muscle stiffness; difficulty walking; tripping and/or falling; hand weakness; twitching in one's aims, shoulders or tongue; inappropriate crying, laughing or yawning; or cognitive and/or behavior problems.

9. The method of claim 1, wherein the A1AT expression or activity is increased in the brain, liver immune system and/or gastrointestinal tract.

10. The method of claim 1, wherein the agent comprises a cell therapy; optionally wherein the cell therapy comprises a stem cell, neuron, glial cell, muscle cell, pancreas cell, hepatocyte, immune cell, bacterial cell, or archaeal cell.

11. The method of claim 10, wherein the cell therapy comprises an engineered cell engineered to express a functional A1AT protein.

12. (canceled)

13. The method of claim 1, wherein the agent comprises an antibody, an enzyme activator, small molecule, or mRNA formulated into lipid nanoparticles.

14. The method of claim 1, wherein the agent comprises a replacement therapy, wherein the replacement therapy comprises:
(i) comprises an A1AT replacement therapy;
(ii) the replacement therapy comprises administration of a functional enzyme, optionally wherein the replacement therapy comprises a purified A1AT protein, optionally wherein the purified A1AT protein comprises Aralast NP;
(iii) the replacement therapy comprises editing a genome of the subject to produce a functional enzyme, optionally wherein the genome editing is Crispr/Cas9-mediated; and/or
(iv) an engineered cell engineered to express a functional enzyme.

15. (canceled)

16. (canceled)

17. (canceled)

18. (canceled)

19. (canceled)

20. The method of claim 1, wherein the method comprises administering to the human subject an agent comprising a pharmaceutically acceptable vehicle and exogenous antibody or binding fragment thereof that binds selectively to all or part of mutant and/or endogenous A1AT, wherein administering the antibody or binding fragment thereof treats or prevents ALS or a symptom thereof.

21. (canceled)

22. The method of claim 20, wherein the antibody is a monoclonal, polyclonal, chimeric or humanized antibody, the antibody binds at least 5 contiguous amino acids of a serpinA1 polypeptide, and/or the antibody binds at least 5 contiguous amino acids of a serpinA1 variant polypeptide expressed by the subject.

23. (canceled)

24. (canceled)

25. The pharmaceutical composition of claim 2, where the pharmaceutical composition comprises (i) an enzyme activator that increases A1AT expression or activity when administered to a human subject in an amount effective to treat or prevent ALS or a symptom thereof; (ii) a small molecule having a molecular weight less than about 5 kiloDaltons in an amount effective to increase A1AT expression or activity when administered to a human subject; (iii) mRNA formulated into a lipid nanoparticle in an amount effective to increase A1AT expression or activity when administered to a human subject; (iv) an antibody in an amount effective to increase A1AT expression or activity when administered to a human subject; (v) pharmaceutically acceptable vehicle and an effective amount of an antibody or binding fragment thereof that binds selectively to a A1AT wild-type polypeptide or A1AT variant polypeptide; (vi) a pharmaceutically acceptable vehicle and an effective amount of an inhibitory nucleic acid thereof that reduces a level of a serpinA1 variant polypeptide; (vii) a pharmaceutically acceptable vehicle and an effective amount of engineered bacteria to reduce a level of a serpinA1 variant polypeptide in a human subject; and/or (viii) a pharmaceutically acceptable vehicle and an effective amount of engineered bacteria to reduce serine protease activity in the gastrointestinal tract of a human subject; and
optionally for (iv) or (v) wherein (a) the antibody binds at least 5 contiguous amino acids of serpinA1 polypeptide; (b) the antibody binds at least 5 contiguous amino acids of a serpinA1 variant polypeptide expressed by the subject; and/or (c) the antibody reduces serpinA1 activity in a human subject.

26. (canceled)

27. (canceled)

28. (canceled)

29. (canceled)

30. (canceled)

31. (canceled)

32. (canceled)

33. (canceled)

34. (canceled)

35. (canceled)

36. The method of claim 1, wherein the agent comprises an autophagy activator, a mitophagy activator, a neutrophil elastase inhibitor, or a combination thereof, optionally wherein:
a. the autophagy activator is independently selected from the group consisting of: carbamazepine (Tegretol), Akebia saponin D, metformin, rapamycin, resveratrol, retinoic acid, and valproic acid;
b. the mitophagy activators is independently selected from the group consisting of celastrol (Tripterin), Urolithin A; and
c. the neutrophil elastase inhibitor is independently selected from the group of ursolic acid, Sivelestat, Alvelestat, sirtinol, and triterpene derived compounds.

37. The method of claim 1, wherein the agent comprises an effective amount of Alvelestat.

38. The method of claim 37, comprising an oral dose of Alvelestat, wherein the dose administered is at least about 120 mg, suitable to be administered one or more times per day.

39. The method of claim 1, wherein the agent comprises an alpha-tocopherol (Vitamin E) analog.

40. The method of claim 1, wherein the agent comprises a triterpene.

41. The method of claim 40, wherein the triterpene administered comprises (i) ursodiol or derivative; (ii) ursolic acid or derivative; and/or (iii) a pentacyclic triterpenoid or derivative.

42. (canceled)

43. (canceled)

44. (canceled)

45. A method of identifying a compound capable of modulating a serine protease inhibitor activity, comprising the steps of (a) identifying one or more functional groups capable of interacting with a serpinA1; and (b) identifying a scaffold which is capable of orienting the one or more functional groups identified in (a) in a suitable orientation for interacting with the serpinA1.

46. A pharmaceutical composition comprising the compound identified in claim 45 in an amount effective to increase serine protease inhibitor activity when administered to a human subject.

47. The method of claim 45, wherein the serpinA1 comprises a variant serpinA1 polypeptide.

48. The method of claim 1, further comprising a method for identifying a subject suitable for treatment of ALS comprising:
a. measuring a first concentration of an alpha-1 anti-trypsin in a biological sample from the subject, optionally wherein the biological sample comprises cerebrospinal fluid, blood, urine gastrointestinal or fecal matter, saliva, tear, or tissue;
b. measuring a second concentration of a neurofilament in the sample; and
c. determining a value comprising the ratio of the first concentration to the second concentration, wherein the value above a threshold value selects the subject for treatment.

49. (canceled)

50. The method of claim 48, wherein the subject has an impaired liver activity or function.

51. The method of claim 50, wherein the method of determining impaired liver activity comprises the step of measuring a steroid hormone level, bile acid production, or production of a cholesterol, lipid, HDL, LDL, triglyceride or cytokine.

52. The method of claim 1, further comprising a biomarker assay for diagnosis of ALS in a human subject, comprising (i) means for determining an insufficient level of serpinA1 protein in the subject; (ii) means for determining a variant serpinA1 protein in the subject; (iii) means for determining serpinA1 protein aggregates in the subject; (iv) means for determining serpinA1-neurofiliment protein aggregates in the subject; and/or (v) means for determining trypsin cleavage products in the subject.

53. (canceled)

54. (canceled)

55. (canceled)

56. (canceled)

57. A method for identifying a human subject for treatment of ALS comprising using the biomarker assay of claim 52 with a biological sample from a subject, and determining a predictive value, which if greater than a certain threshold value identifies the subject as suitable for treatment, optionally wherein the biological sample comprises blood, plasma, derived from peripheral blood mononuclear cell or collected from brain tissue, muscle, liver tissue, or gastrointestinal tract tissue.

58. (canceled)

59. The method of claim 1, comprising administering (i) an agent that increases alpha-1 anti-trypsin (A1AT) activity to the subject wherein the subject is characterized as having hypofunctional alpha-1-anti-trypsin activity; and/or (ii) an agent that decreases serine protease activity to the subject wherein the subject is characterized as having hypofunctional alpha-1-anti-trypsin activity.

60. (canceled)

61. The method of claim 59, wherein the subject has (i) a level of serpinA1 function less than a reference level in an organ or tissue; and/or (ii) the subject has a level of serine protease activity greater than a reference level in an organ or tissue.

62. (canceled)

63. The method of claim 61, wherein the organ or tissue is blood, cerebrospinal fluid, muscle, or gastrointestinal tract.

64. The method of claim 59, comprising the step of administering to the subject an effective amount of an agent that modulates an antitrypsin pathway; optionally wherein the agent is orally delivered, inhaled, infused intravenously, applied transdermal or injected intradermally, intramuscularly, intravenously, or intracranially.

65. The method of claim 64, wherein the agent comprises Prolastin-C, Aralast NP, Zemaira, Glassia or Trypsone.

66. (canceled)
